(19) United States
(12) Patent Application Publication
(10) Pub. No.: US 2022/0071938 A1
(43) Pub. Date: Mar. 10, 2022

(54) TREATMENT OF HYPERAMMONEMIA IN PATIENTS WITH RENAL INSUFFICIENCY

(71) Applicant: RECORDATI S.p.A., Milano (IT)

(72) Inventors: Marco BARCHIELLI, Milano (IT); Roberto GOMENI, Milano (IT)

(21) Appl. No.: 17/506,559

(22) Filed: Oct. 20, 2021

Related U.S. Application Data
(63) Continuation of application No. 16/851,999, filed on Apr. 17, 2020.
(60) Provisional application No. 62/851,339, filed on May 22, 2019.

(51) Int. Cl. A61K 31/197; A61P 3/12

(52) U.S. Cl. CPC A61K 31/197; A61P 3/12

(57) ABSTRACT

Disclosed herein are methods of for treating hyperammonemia in a patient having renal impairment, comprising administering carglumic acid to said patient at a reduced daily dosage.

### CROSS-REFERENCE TO RELATED APPLICATIONS

This application is a continuation of U.S. application Ser. No. 16/851,999 filed Apr. 17, 2020, which claims priority under 35 U.S.C. 119(c) to U.S. Provisional Application Ser. No. 62/851,339, filed May 22, 2019, the contents of each of which are herein incorporated by reference in their entireties.

### BACKGROUND OF THE INVENTION

Hyperammonemia is a metabolic disorder characterized by an excess of ammonia in the blood and can be treated using CARBAGLU® (carglumic acid).

Carglumic acid is N-carbamoyl-L-glutamic acid or (2S)-2-(carbamoylamino) pentanedioic acid, with a molecular formula of C₆H₁₀N₂O₅ and a molecular weight of 190.16. The structural formula of carglumic acid is:

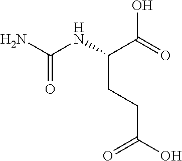 [SMILES: NC(=O)N[C@@H](CCC(=O)O)C(=O)O]

Carglumic acid is marketed under the trade name CARBAGLU® (carglumic acid), which is a tablet for oral suspension containing 200 mg of carglumic acid. A pharmaceutical composition can comprise carglumic acid and one or more inactive ingredients including sodium, hypromellose, microcrystalline cellulose, silica colloidal anhydrous, sodium lauryl sulfate, and sodium stearyl fumarate. CARBAGLU® (carglumic acid) is approved and marketed in the European Union for the treatment of hyperammonemia in patients due to NAGS deficiency and due to propionic acidemia (PA), methymalonic acidemia (MMA) and isovaleric acidemia (IVA). In the United States, CARBAGLU® (carglumic acid) is approved and marketed for acute hyperammonemia in patients with NAGS deficiency and maintenance therapy for chronic hyperammonemia in NAGS deficiency patients.

The current recommended initial and subsequent oral daily dosing regimen of CARBAGLU® (carglumic acid) for the treatment of acute hyperammonemia due to NAGS deficiency in the adult and pediatric population is 100 mg/kg/day to 250 mg/kg/day divided in two to four doses. The current recommended oral daily dosing regimen of CARBAGLU® (carglumic acid) for the maintenance treatment of chronic hyperammonemia due to NAGS deficiency in the adult and pediatric population is 10 mg/kg/day to 100 mg/kg/day divided in two to four doses.

It has recently been found that carglumic acid is predominantly excreted through the kidneys. As such, it is critical that patients with renal insufficiency be dosed differently than patients with normal renal functions. The present disclosure addresses this need by providing methods for treating patients with moderate, severe, and end stage renal disease for acute and/or chronic hyperammonemia with carglumic acid.

### SUMMARY OF THE INVENTION

Disclosed herein is a method for treating hyperammonemia in a patient in need thereof, comprising: identifying the patient as having moderate renal impairment; and administering carglumic acid to the patient at a daily dosage of 5 mg/kg to 125 mg/kg. In some cases, the patient has a glomerular filtration rate (GFR) of 30-59 mL/min/1.73 m².

In some cases, the hyperammonemia is acute hyperammonemia. In some cases, the hyperammonemia is chronic hyperammonemia In some cases, the hyperammonemia is hyperammonemia due to NAGS deficiency, MMA, PA, or IVA. In some cases, the hyperammonemia is acute hyperammonemia due to NAGS deficiency, MMA, PA, or IVA. In some cases, the hyperammonemia is chronic hyperammonemia due to NAGS deficiency, MMA, PA, or IVA.

In some cases, for patients with moderate renal impairment, the daily dosage of carglumic acid for acute hyperammonemia is about 50 mg/kg to about 125 mg/kg. In some cases, for patients with moderate renal impairment, the daily dosage of carglumic acid for acute hyperammonemia is about 50 mg/kg to about 95 mg/kg. In some cases, for patients with moderate renal impairment, the daily dosage of carglumic acid for acute hyperammonemia is about 50 mg/kg to about 90 mg/kg. In some cases, for patients with moderate renal impairment, the daily dosage of carglumic acid for chronic hyperammonemia is about 10 mg/kg to about 50 mg/kg.

Disclosed herein is a method for treating hyperammonemia in a patient in need thereof, comprising: identifying the patient as having severe renal impairment; and administering carglumic acid to the patient at a daily dosage of 2 mg/kg to 60 mg/kg. In some cases, the patient has a glomerular filtration rate (GFR) of 15 to 29 mL/min/1.73 m².

In some cases, the hyperammonemia is acute hyperammonemia. In some cases, the hyperammonemia is chronic hyperammonemia In some cases, the hyperammonemia is hyperammonemia due to NAGS deficiency, MMA, PA, or IVA. In some cases, the hyperammonemia is acute hyperammonemia due to NAGS deficiency, MMA, PA, or IVA. In some cases, the hyperammonemia is chronic hyperammonemia due to NAGS deficiency, MMA, PA, or IVA.

In some cases, for patients with severe renal impairment, the daily dosage of carglumic acid for acute hyperammonemia is about 15 mg/kg to about 60 mg/kg. In some cases, for patients with severe renal impairment, the daily dosage of carglumic acid for chronic hyperammonemia is about 2 mg/kg to about 25 mg/kg.

Disclosed herein is a method for treating hyperammonemia in a patient in need thereof, comprising: identifying the patient as having end stage renal disease; and administering carglumic acid to the patient at a daily dosage of 2 mg/kg to 60 mg/kg. In some cases, the patient has a glomerular filtration rate (GFR) of less than 15 mL/min/1.73 m².

In some cases, the hyperammonemia is acute hyperammonemia. In some cases, the hyperammonemia is chronic hyperammonemia In some cases, the hyperammonemia is hyperammonemia due to NAGS deficiency, MMA, PA, or IVA. In some cases, the hyperammonemia is acute hyperammonemia due to NAGS deficiency, MMA, PA, or IVA. In some cases, the hyperammonemia is chronic hyperammonemia due to NAGS deficiency, MMA, PA, or IVA.

In some cases, for patients with severe renal impairment, the daily dosage of carglumic acid for acute hyperammonemia is about 15 mg/kg to about 60 mg/kg. In some cases, for patients with severe renal impairment, the daily dosage of carglumic acid for chronic hyperammonemia is about 2 mg/kg to about 25 mg/kg.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 shows the ammonia response for 13 NAGS deficiency patients at baseline and after treatment with carglumic acid.

FIG. 2 shows the box and whisker plot of ratio to baseline in plasma ammonia levels over the first 48 hours after treatment administration (by blocks of 12 hours)-FAS (plasma NH₃ data censored at HD/HF/PD initiation) (FAS=full analysis set; HD=hemodialysis; HF=hemofiltration; NH₃=ammonia; PD=peritoneal dialysis; Ratio to baseline was calculated as maximum NH₃ level during the given time period divided by the last NH₃ value prior to treatment initiation; Dotted line=NH₃ level similar as baseline (ratio=1); box limits=Q1 and Q3; ∘=mean; line within box=median; lines outside box=minimum and maximum values).

FIG. 3A shows the mean plasma NH₃ reduction in 12-hour periods; FIG. 3B shows the mean plasma NH₃ reduction in 24-hour periods (PMS Population).

FIG. 4A shows the rate of reduction of plasma NH₃ comparing the 3 treatment groups in patients with baseline plasma NH₃ levels over 100 micromol/L in non-neonate or 150 micromol/L in neonate; FIG. 4B shows the logarithm of the same rates.

FIG. 5A shows the rate of reduction of plasma NH₃ comparing the 3 treatment groups in PMS patients; FIG. 5B shows the logarithm of the same rates.

FIG. 6 shows the expected median carglumic acid concentration profiles in a population of normal subjects and in a population of subjects with mild, moderate, severe, and end stage renal impairment (Age: 20 days; body weight: 3.5 kg).

FIG. 7 shows the expected median carglumic acid concentration profiles in a population of normal subjects and in a population of subjects with mild, moderate, severe, and end stage renal impairment (Age: 1 year; body weight: 9.5 kg).

FIG. 8 shows the expected median carglumic acid concentration profiles in a population of normal subjects and in a population of subjects with mild, moderate, severe, and end stage renal impairment (Age: 15 years; body weight: 50 kg).

FIG. 9 shows the Carglumic Acid Box Plots of Dose Normalized Plasma Cmax Following Single-Oral Administration of Carbaglu in Subjects Renal Impairment and Normal Renal Function. The dashed line is the median; the solid line is the arithmetic mean. The ends of the “box” are the 25th and 75th percentiles. The whiskers show the lowest data value still within 1.5 interquartile range (IQR) of the lower quartile, and the highest value still within 1.5 IQR of the upper quartile, where IQR is the interquartile range (the difference between the third and first quartiles, the middle 50%). Data values that do not fall between the whiskers are plotted as outliers (markers outside of the whiskers).

FIG. 10 shows Carglumic Acid Box Plots of Dose Normalized Plasma AUC0-T Following Single-Oral Administration of Carbaglu in Subjects Renal Impairment and Normal Renal Function. The dashed line is the median; the solid line is the arithmetic mean. The ends of the “box” are the 25th and 75th percentiles. The whiskers show the lowest data value still within 1.5 interquartile range (IQR) of the lower quartile, and the highest value still within 1.5 IQR of the upper quartile, where IQR is the interquartile range (the difference between the third and first quartiles, the middle 50%). Data values that do not fall between the whiskers are plotted as outliers (markers outside of the whiskers).

FIG. 11 shows Carglumic Acid Box Plots of Dose Normalized Plasma AUC_{0-∞} Following Single-Oral Administration of Carbaglu in Subjects Renal Impairment and Normal Renal Function. The dashed line is the median; the solid line is the arithmetic mean. The ends of the “box” are the 25th and 75th percentiles. The whiskers show the lowest data value still within 1.5 interquartile range (IQR) of the lower quartile, and the highest value still within 1.5 IQR of the upper quartile, where IQR is the interquartile range (the difference between the third and first quartiles, the middle 50%). Data values that do not fall between the whiskers are plotted as outliers (markers outside of the whiskers).

FIG. 12 shows FIG. 4: Carglumic Acid Box Plots of Dose Normalized Plasma CL_{TOT} Following Single-Oral Administration of Carbaglu in Subjects Renal Impairment and Normal Renal Function. The dashed line is the median; the solid line is the arithmetic mean. The ends of the “box” are the 25th and 75th percentiles. The whiskers show the lowest data value still within 1.5 interquartile range (IQR) of the lower quartile, and the highest value still within 1.5 IQR of the upper quartile, where IQR is the interquartile range (the difference between the third and first quartiles, the middle 50%). Data values that do not fall between the whiskers are plotted as outliers (markers outside of the whiskers).

FIG. 13A-B shows Mean Plasma Concentration-Time Profile of Carglumic Acid in Subjects with Renal Impairment and Normal Renal Function Following Single-Oral Administration of Carbaglu (Linear Scale-Panel A) (Semi-Logarithmic Scale—Panel B).

FIG. 14 shows Carglumic Acid Box Plots of Urine CLR(0-T) Following Single-Oral Administration of Carbaglu in Subjects Renal Impairment and Normal Renal Function. In the Box plot, the dashed line is the median; the solid line is the arithmetic mean. The ends of the “box” are the 25th and 75th percentiles. The whiskers show the lowest data value still within 1.5 interquartile range (IQR) of the lower quartile, and the highest value still within 1.5 IQR of the upper quartile, where IQR is the interquartile range (the difference between the third and first quartiles, the middle 50%). Data values that do not fall between the whiskers are plotted as outliers (markers outside of the whiskers).FIG. 15 shows Mean Cumulative Amount Urine Excreted-Midpoint Profile of Carglumic Acid in Subjects with Renal Impairment and Normal Renal Function Following Single-Oral Administration of Carbaglu (Linear Scale).

FIG. 16A-C shows Individual Plasma Time-Concentrations of Carglumic Acid in Healthy Subjects Following a Single IV Administration of a Dose of 3.5 (Panel A), 5.0 (Panel B) or 7.5 (Panel C) mg/kg over 20 Minutes.

FIG. 17A-B shows mean Carbaglu plasma concentrations (±SD) vs. nominal time by treatment and dose in log-linear scale; in the range of time 0 to 72 hours (Panel A) and 0 to 12 hours (Panel B).

FIG. 18A-B shows mean Carbaglu plasma concentrations (±SD) vs. nominal time in group 1, 2, and 3 (Panel A) and 4 and 5 (Panel B) in log-linear scale, in the range of time 0 to 48 hours.

### DETAILED DESCRIPTION OF THE INVENTION

### Definitions

It is to be understood that unless otherwise indicated the present invention is not limited to specific formulation components, drug delivery systems, manufacturing techniques, administration steps, or the like, as such may vary. In this regard, unless otherwise stated, a reference to a compound or component includes the compound or component by itself, as well as the compound or component in combination with other compounds or components, such as mixtures of compounds.

As used herein, the singular forms “a,” “an,” and “the” include plural referents unless the context clearly dictates otherwise. Thus, for example, reference to “an antiarrhythmic pharmaceutical agent” includes not only a single active agent but also a combination or mixture of two or more different active agents.

Reference herein to “one embodiment,” “one version,” or “one aspect” shall include one or more such embodiments, versions or aspects, unless otherwise clear from the context.

The term “about” in relation to a reference numerical value can include a range of values plus or minus 10% from that value. For example, the amount “about 10” includes amounts from 9 to 11, including the reference numbers of 9, 10, and 11. The term “about” in relation to a reference numerical value can also include a range of values plus or minus 10%, 9%, 8%, 7%, 6%, 5%, 4%, 3%, 2%, or 1% from that value.

As used herein, the terms “treating” and “treatment” can refer to reduction in severity and/or frequency of symptoms, elimination of symptoms and/or underlying cause, reduction in likelihood of the occurrence of symptoms and/or underlying cause, and/or remediation of damage. Thus, “treating” a patient with an active agent as provided herein includes prevention of a particular condition, disease, or disorder in a susceptible individual as well as treatment of a clinically symptomatic individual.

The term “eGFR,” as used herein, refers to estimated glomerular filtration rate, and is an indicator of renal function. eGFR is a mathematically derived entity based on a patient's serum creatinine level, age, sex and race, and can be measured and calculated using the MDRD equation:

eGFR=175×(S_{Cr})^{-1.154}×(age)^{-0.203}×0.742[if female]×1.212[if Black]

wherein the unit of eGFR, S_{cr} (standardized serum creatinine), and age is mL/min/1.73 m², mg/dL, and years, respectively. For example, a healthy subject with normal renal function has an eGFR of at least about 90 mL/min/1.73 m²; a mild renal impairment subject has an eGFR of about 60-89 mL/min/1.73 m²; a moderate renal impairment subject has an eGFR of about 30-59 mL/min/1.73 m²; and a severe renal impairment subject has an eGFR of 15 to 29 mL/min/1.73 m². A subject with end stage renal disease, also referred to as end stage renal impairment herein, has an eGFR of less than 15 mL/min/1.73 m².

The dosage unit mg/kg, as used herein, refers to the milligrams of the drug taken by per kg body weight of the patient. For example, if the daily dosage of carglumic acid is 100 mg/kg, a patient with a body weight of 80 kg should take 8,000 mg of carglumic acid (equals to 40 CARBAGLU® tablets).

The term “comparable patient” is used in comparison to another patent with a renal impairment. The comparable patient does not have the renal impairment but has the same body weight as the patent with the renal impairment.

### Mechanism

Carglumic acid is a synthetic structural analogue of N-acetylglutamate (NAG) which is produced from glutamate and acetyl-CoA in a reaction catalyzed by N-acetylglutamate synthase (NAGS), a mitochondrial liver enzyme. NAG acts as the essential allosteric activator of Carbamoyl Phosphate Synthetase 1 (CPS 1), a mitochondrial liver enzyme which catalyzes the first reaction of the urea cycle. The urea cycle, whose role is the disposition of ammonia, includes a series of biochemical reactions in the liver resulting in the conversion of ammonia into urea, which is then excreted through the urine. Carglumic acid can act as a CPS1 activator in patients with NAGS deficiency, thereby removing the block in the urea cycle and facilitating ammonia detoxification and urea production.

In PA and MMA, accumulation of propionyl-CoA and methylmalonyl-CoA in liver cell mitochondria inhibits NAGS activity, leading to secondary deficiency of NAG and hyperammonemia. Propionyl-CoA and methylmalonyl-CoA also inhibit the pathway by depleting hepatic acetyl-CoA, which is required for NAG synthesis. Carglumic acid can act as a CPS1 activator and improves or restores the function of the urea cycle, facilitating ammonia detoxification and urea production.

Carglumic acid can be used for treatment of some organic acidemias (OAs): isovaleric acidemia (IA), methylmalonic acidemia (MMA), and propionic acidemia (PA). Patients with these diseases produce defective versions of the same enzymes, isovaleryl-coA dehydrogenase, methylmalonyl-coA mutase and propionyl-coA carboxylase, respectively. The lack of activity of these enzymes, involved in protein and fat metabolism and in branched-chain amino acid catabolic pathway, causes a build-up of acids (isovaleric, methylmalonic and propionic acid) and ammonia in the blood. An inhibition of NAGS activity either through a direct inhibition caused by the accumulation of organic acids, or through the depletion of acetyl-CoA or coenzyme A itself, both can occur in OA.

The treatment of acute hyperammonemia can be an emergency, due to the potential risk for irreversible brain damage or death. Under these conditions, the patients need to be treated immediately in case of presumptive evidence of NAGS or other enzyme deficiencies.

In patients with NAGS deficiency, carglumic acid can induce a rapid normalization of plasma ammonia levels, usually within 24 hours. When the treatment was instituted before any permanent brain damage, patients can exhibit normal growth and psychomotor development. In patients with PA, MMA, or IVA (neonates and non-neonates), the treatment with carglumic acid can induce a quick decrease of ammonia plasma levels, reducing the risk of neurological complications.

### Indications

Carglumic acid is intended for the treatment of hyperammonemia in patients with different metabolic diseases, including but not limited to N-acetylglutamate synthase (NAGS) deficiency, methylmalonic acidemia (MMA), propionic acidemia (PA), or isovaleric acidemia (IA). Hyperammonemia can be due to ornithine transcarbamylase deficiency, hyperinsulinism-hyperammonemia syndrome (glutamate dehydrogenase 1), hyperornithinemia-hyperammonemia-homocitrullinuria, N-acetylglutamate synthase (NAGS) deficiency, carbamoyl phosphate synthase I deficiency (carbamoyl phosphate synthase I), hyperlysinuria with hyperammonemia, methylmalonic acidemia (methylmalonic aciduria), isovaleric acidemia (isovaleric aciduria), propionic acidemia (propionic aciduria), carnitine palmitoyltransferase II deficiency, or newborn transient hyperammonemia. In some cases, the hyperammonemia is due to N-acetylglutamate synthase (NAGS) deficiency, methylmalonic acidemia, isovaleric acidemia, or propionic acidemia.

Carglumic acid can be used for treating hyperammonemia due to the deficiency of the hepatic enzyme N-acetylglutamate synthase (NAGS). In some cases, carglumic acid can be used as an adjunctive therapy in pediatric and adult patients for the treatment of acute hyperammonemia. During acute hyperammonemic episodes, carglumic acid can be administered with other ammonia lowering therapies, such as alternate pathway medications, hemodialysis, and dietary protein restriction.

Carglumic acid can be used for treating chronic hyperammonemia due to the deficiency of the hepatic enzyme N-acetylglutamate synthase (NAGS). For example, carglumic acid can be used as maintenance therapy in pediatric and adult patients for the treatment of chronic hyperammonemia due to deficiency of the hepatic enzyme N-acetylglutamate synthase (NAGS). During maintenance therapy, carglumic acid can be administered with other ammonia lowering therapies and protein restriction.

Carglumic acid can be used for treating hyperammonemia due to methylmalonic acidemia (MMA). In some cases, carglumic acid can be used as adjunctive therapy for the treatment of acute hyperammonemia due to methylmalonic acidemia. During acute hyperammonemic episodes, carglumic acid can be administered with standard of care for metabolic decompensation.

Carglumic acid can be used for treating hyperammonemia due to propionic acidemia (PA). In some cases, carglumic acid can be used as adjunctive therapy for the treatment of acute hyperammonemia due to propionic acidemia. During acute hyperammonemic episodes, carglumic acid can be administered with standard of care for metabolic decompensation.

Carglumic acid can also be used for treating hyperammonemia due to isovaleric acidemia (IA). In some cases, carglumic acid can be used as adjunctive therapy for the treatment of acute hyperammonemia due to isovaleric acidemia. During acute hyperammonemic episodes, carglumic acid can be administered with standard of care for metabolic decompensation.

Carglumic acid can be used for treating hyperammonemia in patients with a suspected or confirmed diagnosis of NAGS deficiency, MMA, PA, or IVA.

### Dosages

Patients without Renal Impairment

Disclosed herein are methods for treating hyperammonemia in a patient in need thereof, comprising: a) identifying said patient as not having renal impairment; and b) administering carglumic acid to said patient at a daily dosage of 100 mg/kg to 250 mg/kg. In some cases, the hyperammonemia is acute hyperammonemia due to NAGS deficiency, MMA, PA, or IVA. In some cases, the hyperammonemia is chronic hyperammonemia due to NAGS deficiency, MMA, PA, or IVA.

For patients without renal impairment, the daily dosage of carglumic acid for acute hyperammonemia can be about 100 mg/kg to about 250 mg/kg. The daily dosage of carglumic acid for chronic hyperammonemia can be about 10 mg/kg to about 100 mg/kg. The daily dosage of carglumic acid can be at least about 10 mg/kg. For patients without renal impairment, the daily dosage of carglumic acid can be at least about 100 mg/kg. For patients without renal impairment, the daily dosage of carglumic acid can be at most about 250 mg/kg. For patients without renal impairment, the daily dosage of carglumic acid can be about 100 mg/kg to about 150 mg/kg, about 100 mg/kg to about 200 mg/kg, about 100 mg/kg to about 250 mg/kg, about 150 mg/kg to about 200 mg/kg, about 150 mg/kg to about 250 mg/kg, or about 200 mg/kg to about 250 mg/kg. For patients without renal impairment, the daily dosage of carglumic acid can be about 100 mg/kg, about 150 mg/kg, about 200 mg/kg, or about 250 mg/kg. For patients without renal impairment, the daily dosage of carglumic acid can be divided into 2 to 4 doses and/or rounded to the nearest 100 mg (i.e., half of a CARBAGLU® tablet).

Patients with Mild Renal Impairment

Disclosed herein are methods for treating hyperammonemia in a patient in need thereof, comprising: a) identifying said patient as having mild renal impairment; and b) administering carglumic acid to said patient at a daily dosage of 100 mg/kg to 250 mg/kg. In some cases, the patient has a glomerular filtration rate (GFR) of 60-89 mL/min/1.73 m².

In some cases, the hyperammonemia is acute hyperammonemia due to NAGS deficiency, MMA, PA, or IVA. In some cases, the hyperammonemia is chronic hyperammonemia due to NAGS deficiency, MMA, PA, or IVA.

For patients with mild renal impairment, the daily dosage of carglumic acid for acute hyperammonemia can be about 100 mg/kg to about 250 mg/kg. For patients with mild renal impairment, the daily dosage of carglumic acid for chronic hyperammonemia can be about 10 mg/kg to about 100 mg/kg. For patients with mild renal impairment, the daily dosage of carglumic acid can be at least about 10 mg/kg. For patients with mild renal impairment, the daily dosage of carglumic acid can be at least about 100 mg/kg. For patients with mild renal impairment, the daily dosage of carglumic acid can be at most about 250 mg/kg. For patients with mild renal impairment, the daily dosage of carglumic acid can be about 100 mg/kg to about 125 mg/kg, 100 mg/kg to about 150 mg/kg, 100 mg/kg to about 175 mg/kg, about 100 mg/kg to about 200 mg/kg, 100 mg/kg to about 225 mg/kg, about 100 mg/kg to about 250 mg/kg, about 150 mg/kg to about 175 mg/kg, about 150 mg/kg to about 200 mg/kg, about 150 mg/kg to about 225 mg/kg, about 150 mg/kg to about 250 mg/kg, about 175 mg/kg to about 200 mg/kg, about 175 mg/kg to about 225 mg/kg, about 175 mg/kg to about 250 mg/kg, about 200 mg/kg to about 225 mg/kg, or about 200 mg/kg to about 250 mg/kg. For patients with mild renal impairment, the daily dosage of carglumic acid can be about 100 mg/kg, about 125 mg/kg, about 150 mg/kg, about 175 mg/kg, about 200 mg/kg, about 225 mg/kg, or about 250 mg/kg. For patients with mild renal impairment, the daily dosage of carglumic acid can be divided into 2 to 4 doses and/or rounded to the nearest 100 mg (i.e., half of a CARBAGLU® tablet).

Patients with Moderate Renal Impairment

Disclosed herein is a method for treating hyperammonemia in a patient in need thereof, comprising: a) identifying the patient as having moderate renal impairment; and b) administering carglumic acid to the patient at a daily dosage of 5 mg/kg to 125 mg/kg. In some cases, the patient has a glomerular filtration rate (GFR) of 30-59 mL/min/1.73 m².

In some cases, the hyperammonemia is acute hyperammonemia. In some cases, the hyperammonemia is chronic hyperammonemia In some cases, the hyperammonemia is hyperammonemia due to NAGS deficiency, MMA, PA, or IVA. In some cases, the hyperammonemia is acute hyperammonemia due to NAGS deficiency, MMA, PA, or IVA. In some cases, the hyperammonemia is chronic hyperammonemia due to NAGS deficiency, MMA, PA, or IVA.

In some cases, for patients with moderate renal impairment, the daily dosage of carglumic acid for acute hyperammonemia is about 50 mg/kg to about 125 mg/kg. In some cases, for patients with moderate renal impairment, the daily dosage of carglumic acid for acute hyperammonemia is about 50 mg/kg to about 95 mg/kg. In some cases, for patients with moderate renal impairment, the daily dosage of carglumic acid for acute hyperammonemia is about 50 mg/kg to about 90 mg/kg. In some cases, for patients with moderate renal impairment, the daily dosage of carglumic acid for acute hyperammonemia is about 30 mg/kg to about 50 mg/kg, 30 mg/kg to about 70 mg/kg, 30 mg/kg to about 90 mg/kg, 30 mg/kg to about 125 mg/kg, 40 mg/kg to about 50 mg/kg, 40 mg/kg to about 60 mg/kg, 40 mg/kg to about 70 mg/kg, 40 mg/kg to about 80 mg/kg, 40 mg/kg to about 90 mg/kg, 40 mg/kg to about 100 mg/kg, 40 mg/kg to about 125 mg/kg, 50 mg/kg to about 60 mg/kg, 50 mg/kg to about 70 mg/kg, 50 mg/kg to about 80 mg/kg, 50 mg/kg to about 90 mg/kg, 50 mg/kg to about 100 mg/kg, 50 mg/kg to about 125 mg/kg, 60 mg/kg to about 70 mg/kg, 60 mg/kg to about 80 mg/kg, 60 mg/kg to about 90 mg/kg, 60 mg/kg to about 100 mg/kg, 60 mg/kg to about 125 mg/kg, 70 mg/kg to about 80 mg/kg, 70 mg/kg to about 90 mg/kg, 70 mg/kg to about 100 mg/kg, 70 mg/kg to about 125 mg/kg, 80 mg/kg to about 90 mg/kg, 90 mg/kg to about 100 mg/kg, 90 mg/kg to about 125 mg/kg, or 100 mg/kg to about 125 mg/kg.

In some cases, for patients with moderate renal impairment, the daily dosage of carglumic acid for chronic hyperammonemia is about 10 mg/kg to about 50 mg/kg. In some cases, for patients with moderate renal impairment, the daily dosage of carglumic acid for chronic hyperammonemia is about 5 mg/kg to about 10 mg/kg, 5 mg/kg to about 15 mg/kg, 5 mg/kg to about 20 mg/kg, 5 mg/kg to about 25 mg/kg, 5 mg/kg to about 30 mg/kg, 5 mg/kg to about 35 mg/kg, 5 mg/kg to about 40 mg/kg, 5 mg/kg to about 45 mg/kg, 5 mg/kg to about 50 mg/kg, 5 mg/kg to about 55 mg/kg, 5 mg/kg to about 60 mg/kg, 10 mg/kg to about 15 mg/kg, 10 mg/kg to about 20 mg/kg, 10 mg/kg to about 25 mg/kg, 10 mg/kg to about 30 mg/kg, 10 mg/kg to about 35 mg/kg, 10 mg/kg to about 40 mg/kg, 10 mg/kg to about 45 mg/kg, 10 mg/kg to about 50 mg/kg, 10 mg/kg to about 55 mg/kg, 10 mg/kg to about 60 mg/kg, 20 mg/kg to about 30 mg/kg, 20 mg/kg to about 35 mg/kg, 20 mg/kg to about 40 mg/kg, 20 mg/kg to about 45 mg/kg, 20 mg/kg to about 50 mg/kg, 20 mg/kg to about 55 mg/kg, 20 mg/kg to about 60 mg/kg, 30 mg/kg to about 35 mg/kg, 30 mg/kg to about 40 mg/kg, 30 mg/kg to about 45 mg/kg, 30 mg/kg to about 50 mg/kg, 30 mg/kg to about 55 mg/kg, 30 mg/kg to about 60 mg/kg, 40 mg/kg to about 45 mg/kg, 40 mg/kg to about 50 mg/kg, 40 mg/kg to about 55 mg/kg, 40 mg/kg to about 60 mg/kg, 45 mg/kg to about 50 mg/kg, 45 mg/kg to about 55 mg/kg, 45 mg/kg to about 60 mg/kg, 50 mg/kg to about 55 mg/kg, 50 mg/kg to about 60 mg/kg, or 50 mg/kg to about 60 mg/kg.

Patients with Severe Renal Impairment

Disclosed herein is a method for treating hyperammonemia in a patient in need thereof, comprising: a) identifying the patient as having severe renal impairment; and administering carglumic acid to the patient at a daily dosage of 2 mg/kg to 60 mg/kg. In some cases, the patient has a glomerular filtration rate (GFR) of 15 to 29 mL/min/1.73 m².

In some cases, the hyperammonemia is acute hyperammonemia. In some cases, the hyperammonemia is chronic hyperammonemia In some cases, the hyperammonemia is hyperammonemia due to NAGS deficiency, MMA, PA, or IVA. In some cases, the hyperammonemia is acute hyperammonemia due to NAGS deficiency, MMA, PA, or IVA. In some cases, the hyperammonemia is chronic hyperammonemia due to NAGS deficiency, MMA, PA, or IVA.

In some cases, for patients with severe renal impairment, the daily dosage of carglumic acid for acute hyperammonemia is about 15 mg/kg to about 60 mg/kg. In some cases, for patients with severe renal impairment, the daily dosage of carglumic acid for acute hyperammonemia is about 5 mg/kg to about 10 mg/kg, 5 mg/kg to about 15 mg/kg, 5 mg/kg to about 20 mg/kg, 5 mg/kg to about 25 mg/kg, 5 mg/kg to about 30 mg/kg, 5 mg/kg to about 35 mg/kg, 5 mg/kg to about 40 mg/kg, 5 mg/kg to about 45 mg/kg, 5 mg/kg to about 50 mg/kg, 5 mg/kg to about 55 mg/kg, 5 mg/kg to about 60 mg/kg, 5 mg/kg to about 65 mg/kg, 5 mg/kg to about 70 mg/kg, 5 mg/kg to about 75 mg/kg, 10 mg/kg to about 15 mg/kg, 10 mg/kg to about 20 mg/kg, 10 mg/kg to about 25 mg/kg, 10 mg/kg to about 30 mg/kg, 10 mg/kg to about 35 mg/kg, 10 mg/kg to about 40 mg/kg, 10 mg/kg to about 45 mg/kg, 10 mg/kg to about 50 mg/kg, 10 mg/kg to about 55 mg/kg, 10 mg/kg to about 60 mg/kg, 10 mg/kg to about 65 mg/kg, 10 mg/kg to about 70 mg/kg, 10 mg/kg to about 75 mg/kg, 15 mg/kg to about 20 mg/kg, 15 mg/kg to about 25 mg/kg, 15 mg/kg to about 30 mg/kg, 15 mg/kg to about 35 mg/kg, 15 mg/kg to about 40 mg/kg, 15 mg/kg to about 45 mg/kg, 15 mg/kg to about 50 mg/kg, 15 mg/kg to about 55 mg/kg, 15 mg/kg to about 60 mg/kg, 15 mg/kg to about 65 mg/kg, 15 mg/kg to about 70 mg/kg, 15 mg/kg to about 75 mg/kg, 20 mg/kg to about 30 mg/kg, 20 mg/kg to about 35 mg/kg, 20 mg/kg to about 40 mg/kg, 20 mg/kg to about 45 mg/kg, 20 mg/kg to about 50 mg/kg, 20 mg/kg to about 55 mg/kg, 20 mg/kg to about 60 mg/kg, 20 mg/kg to about 65 mg/kg, 20 mg/kg to about 70 mg/kg, 20 mg/kg to about 75 mg/kg, 30 mg/kg to about 35 mg/kg, 30 mg/kg to about 40 mg/kg, 30 mg/kg to about 45 mg/kg, 30 mg/kg to about 50 mg/kg, 30 mg/kg to about 55 mg/kg, 30 mg/kg to about 60 mg/kg, 30 mg/kg to about 65 mg/kg, 30 mg/kg to about 70 mg/kg, 30 mg/kg to about 75 mg/kg, 40 mg/kg to about 45 mg/kg, 40 mg/kg to about 50 mg/kg, 40 mg/kg to about 55 mg/kg, 40 mg/kg to about 60 mg/kg, 40 mg/kg to about 65 mg/kg, 40 mg/kg to about 70 mg/kg, 40 mg/kg to about 75 mg/kg, 50 mg/kg to about 55 mg/kg, 50 mg/kg to about 60 mg/kg, 50 mg/kg to about 60 mg/kg, 50 mg/kg to about 65 mg/kg, 50 mg/kg to about 70 mg/kg, 50 mg/kg to about 75 mg/kg, 60 mg/kg to about 65 mg/kg, 60 mg/kg to about 70 mg/kg, 60 mg/kg to about 75 mg/kg, 65 mg/kg to about 70 mg/kg, or 65 mg/kg to about 75 mg/kg.

In some cases, for patients with severe renal impairment, the daily dosage of carglumic acid for chronic hyperammonemia is about 2 mg/kg to about 25 mg/kg. In some cases, for patients with severe renal impairment, the daily dosage of carglumic acid for chronic hyperammonemia is about 1 mg/kg to about 3 mg/kg, 1 mg/kg to about 7 mg/kg, 1 mg/kg to about 10 mg/kg, 1 mg/kg to about 12 mg/kg, 1 mg/kg to about 15 mg/kg, 1 mg/kg to about 17 mg/kg, 1 mg/kg to about 20 mg/kg, 1 mg/kg to about 22 mg/kg, 1 mg/kg to about 25 mg/kg, 1 mg/kg to about 27 mg/kg, 1 mg/kg to about 30 mg/kg, 2 mg/kg to about 3 mg/kg, 2 mg/kg to about 7 mg/kg, 2 mg/kg to about 10 mg/kg, 2 mg/kg to about 12 mg/kg, 2 mg/kg to about 15 mg/kg, 2 mg/kg to about 17 mg/kg, 2 mg/kg to about 20 mg/kg, 2 mg/kg to about 22 mg/kg, 2 mg/kg to about 25 mg/kg, 2 mg/kg to about 27 mg/kg, 2 mg/kg to about 30 mg/kg, 5 mg/kg to about 7 mg/kg, 5 mg/kg to about 10 mg/kg, 5 mg/kg to about 12 mg/kg, 5 mg/kg to about 15 mg/kg, 5 mg/kg to about 17 mg/kg, 5 mg/kg to about 20 mg/kg, 5 mg/kg to about 22 mg/kg, 5 mg/kg to about 25 mg/kg, 5 mg/kg to about 27 mg/kg, 5 mg/kg to about 30 mg/kg, 7 mg/kg to about 10 mg/kg, 7 mg/kg to about 12 mg/kg, 7 mg/kg to about 15 mg/kg, 7 mg/kg to about 17 mg/kg, 7 mg/kg to about 20 mg/kg, 7 mg/kg to about 22 mg/kg, 7 mg/kg to about 25 mg/kg, 7 mg/kg to about 27 mg/kg, 7 mg/kg to about 30 mg/kg, 10 mg/kg to about 12 mg/kg, 10 mg/kg to about 15 mg/kg, 10 mg/kg to about 17 mg/kg, 10 mg/kg to about 20 mg/kg, 10 mg/kg to about 22 mg/kg, 10 mg/kg to about 25 mg/kg, 10 mg/kg to about 27 mg/kg, 10 mg/kg to about 30 mg/kg, 12 mg/kg to about 15 mg/kg, 12 mg/kg to about 17 mg/kg, 12 mg/kg to about 20 mg/kg, 12 mg/kg to about 22 mg/kg, 12 mg/kg to about 25 mg/kg, 12 mg/kg to about 27 mg/kg, 12 mg/kg to about 30 mg/kg, 15 mg/kg to about 17 mg/kg, 15 mg/kg to about 20 mg/kg, 15 mg/kg to about 22 mg/kg, 15 mg/kg to about 25 mg/kg, 15 mg/kg to about 27 mg/kg, 15 mg/kg to about 30 mg/kg, 17 mg/kg to about 20 mg/kg, 17 mg/kg to about 22 mg/kg, 17 mg/kg to about 25 mg/kg, 17 mg/kg to about 27 mg/kg, 17 mg/kg to about 30 mg/kg, 20 mg/kg to about 22 mg/kg, 20 mg/kg to about 25 mg/kg, 20 mg/kg to about 27 mg/kg, 20 mg/kg to about 30 mg/kg, 22 mg/kg to about 25 mg/kg, 22 mg/kg to about 27 mg/kg, 22 mg/kg to about 30 mg/kg, 25 mg/kg to about 27 mg/kg, 25 mg/kg to about 30 mg/kg, or 27 mg/kg to about 30 mg/kg.

Patients with End Stage Renal Disease

Disclosed herein is a method for treating hyperammonemia in a patient in need thereof, comprising: identifying the patient as having end stage renal disease; and administering carglumic acid to the patient at a daily dosage of 2 mg/kg to 60 mg/kg. In some cases, the patient has a glomerular filtration rate (GFR) of less than 15 mL/min/1.73 m².

In some cases, the hyperammonemia is acute hyperammonemia. In some cases, the hyperammonemia is chronic hyperammonemia In some cases, the hyperammonemia is hyperammonemia due to NAGS deficiency, MMA, PA, or IVA. In some cases, the hyperammonemia is acute hyperammonemia due to NAGS deficiency, MMA, PA, or IVA. In some cases, the hyperammonemia is chronic hyperammonemia due to NAGS deficiency, MMA, PA, or IVA.

In some cases, for patients with end stage renal disease, the daily dosage of carglumic acid for acute hyperammonemia is about 15 mg/kg to about 60 mg/kg. In some cases, for patients with end stage renal disease the daily dosage of carglumic acid for acute hyperammonemia is about 5 mg/kg to about 10 mg/kg, 5 mg/kg to about 15 mg/kg, 5 mg/kg to about 20 mg/kg, 5 mg/kg to about 25 mg/kg, 5 mg/kg to about 30 mg/kg, 5 mg/kg to about 35 mg/kg, 5 mg/kg to about 40 mg/kg, 5 mg/kg to about 45 mg/kg, 5 mg/kg to about 50 mg/kg, 5 mg/kg to about 55 mg/kg, 5 mg/kg to about 60 mg/kg, 5 mg/kg to about 65 mg/kg, 5 mg/kg to about 70 mg/kg, 5 mg/kg to about 75 mg/kg, 10 mg/kg to about 15 mg/kg, 10 mg/kg to about 20 mg/kg, 10 mg/kg to about 25 mg/kg, 10 mg/kg to about 30 mg/kg, 10 mg/kg to about 35 mg/kg, 10 mg/kg to about 40 mg/kg, 10 mg/kg to about 45 mg/kg, 10 mg/kg to about 50 mg/kg, 10 mg/kg to about 55 mg/kg, 10 mg/kg to about 60 mg/kg, 10 mg/kg to about 65 mg/kg, 10 mg/kg to about 70 mg/kg, 10 mg/kg to about 75 mg/kg, 15 mg/kg to about 20 mg/kg, 15 mg/kg to about 25 mg/kg, 15 mg/kg to about 30 mg/kg, 15 mg/kg to about 35 mg/kg, 15 mg/kg to about 40 mg/kg, 15 mg/kg to about 45 mg/kg, 15 mg/kg to about 50 mg/kg, 15 mg/kg to about 55 mg/kg, 15 mg/kg to about 60 mg/kg, 15 mg/kg to about 65 mg/kg, 15 mg/kg to about 70 mg/kg, 15 mg/kg to about 75 mg/kg, 20 mg/kg to about 30 mg/kg, 20 mg/kg to about 35 mg/kg, 20 mg/kg to about 40 mg/kg, 20 mg/kg to about 45 mg/kg, 20 mg/kg to about 50 mg/kg, 20 mg/kg to about 55 mg/kg, 20 mg/kg to about 60 mg/kg, 20 mg/kg to about 65 mg/kg, 20 mg/kg to about 70 mg/kg, 20 mg/kg to about 75 mg/kg, 30 mg/kg to about 35 mg/kg, 30 mg/kg to about 40 mg/kg, 30 mg/kg to about 45 mg/kg, 30 mg/kg to about 50 mg/kg, 30 mg/kg to about 55 mg/kg, 30 mg/kg to about 60 mg/kg, 30 mg/kg to about 65 mg/kg, 30 mg/kg to about 70 mg/kg, 30 mg/kg to about 75 mg/kg, 40 mg/kg to about 45 mg/kg, 40 mg/kg to about 50 mg/kg, 40 mg/kg to about 55 mg/kg, 40 mg/kg to about 60 mg/kg, 40 mg/kg to about 65 mg/kg, 40 mg/kg to about 70 mg/kg, 40 mg/kg to about 75 mg/kg, 50 mg/kg to about 55 mg/kg, 50 mg/kg to about 60 mg/kg, 50 mg/kg to about 60 mg/kg, 50 mg/kg to about 65 mg/kg, 50 mg/kg to about 70 mg/kg, 50 mg/kg to about 75 mg/kg, 60 mg/kg to about 65 mg/kg, 60 mg/kg to about 70 mg/kg, 60 mg/kg to about 75 mg/kg, 65 mg/kg to about 70 mg/kg, or 65 mg/kg to about 75 mg/kg.

In some cases, for patients with end stage renal disease, the daily dosage of carglumic acid for chronic hyperammonemia is about 2 mg/kg to about 25 mg/kg. In some cases, for patients with end stage renal disease, the daily dosage of carglumic acid for chronic hyperammonemia is about 1 mg/kg to about 3 mg/kg, 1 mg/kg to about 7 mg/kg, 1 mg/kg to about 10 mg/kg, 1 mg/kg to about 12 mg/kg, 1 mg/kg to about 15 mg/kg, 1 mg/kg to about 17 mg/kg, 1 mg/kg to about 20 mg/kg, 1 mg/kg to about 22 mg/kg, 1 mg/kg to about 25 mg/kg, 1 mg/kg to about 27 mg/kg, 1 mg/kg to about 30 mg/kg, 2 mg/kg to about 3 mg/kg, 2 mg/kg to about 7 mg/kg, 2 mg/kg to about 10 mg/kg, 2 mg/kg to about 12 mg/kg, 2 mg/kg to about 15 mg/kg, 2 mg/kg to about 17 mg/kg, 2 mg/kg to about 20 mg/kg, 2 mg/kg to about 22 mg/kg, 2 mg/kg to about 25 mg/kg, 2 mg/kg to about 27 mg/kg, 2 mg/kg to about 30 mg/kg, 5 mg/kg to about 7 mg/kg, 5 mg/kg to about 10 mg/kg, 5 mg/kg to about 12 mg/kg, 5 mg/kg to about 15 mg/kg, 5 mg/kg to about 17 mg/kg, 5 mg/kg to about 20 mg/kg, 5 mg/kg to about 22 mg/kg, 5 mg/kg to about 25 mg/kg, 5 mg/kg to about 27 mg/kg, 5 mg/kg to about 30 mg/kg, 7 mg/kg to about 10 mg/kg, 7 mg/kg to about 12 mg/kg, 7 mg/kg to about 15 mg/kg, 7 mg/kg to about 17 mg/kg, 7 mg/kg to about 20 mg/kg, 7 mg/kg to about 22 mg/kg, 7 mg/kg to about 25 mg/kg, 7 mg/kg to about 27 mg/kg, 7 mg/kg to about 30 mg/kg, 10 mg/kg to about 12 mg/kg, 10 mg/kg to about 15 mg/kg, 10 mg/kg to about 17 mg/kg, 10 mg/kg to about 20 mg/kg, 10 mg/kg to about 22 mg/kg, 10 mg/kg to about 25 mg/kg, 10 mg/kg to about 27 mg/kg, 10 mg/kg to about 30 mg/kg, 12 mg/kg to about 15 mg/kg, 12 mg/kg to about 17 mg/kg, 12 mg/kg to about 20 mg/kg, 12 mg/kg to about 22 mg/kg, 12 mg/kg to about 25 mg/kg, 12 mg/kg to about 27 mg/kg, 12 mg/kg to about 30 mg/kg, 15 mg/kg to about 17 mg/kg, 15 mg/kg to about 20 mg/kg, 15 mg/kg to about 22 mg/kg, 15 mg/kg to about 25 mg/kg, 15 mg/kg to about 27 mg/kg, 15 mg/kg to about 30 mg/kg, 17 mg/kg to about 20 mg/kg, 17 mg/kg to about 22 mg/kg, 17 mg/kg to about 25 mg/kg, 17 mg/kg to about 27 mg/kg, 17 mg/kg to about 30 mg/kg, 20 mg/kg to about 22 mg/kg, 20 mg/kg to about 25 mg/kg, 20 mg/kg to about 27 mg/kg, 20 mg/kg to about 30 mg/kg, 22 mg/kg to about 25 mg/kg, 22 mg/kg to about 27 mg/kg, 22 mg/kg to about 30 mg/kg, 25 mg/kg to about 27 mg/kg, 25 mg/kg to about 30 mg/kg, or 27 mg/kg to about 30 mg/kg.

### Additional Dose Adjustments

Disclosed herein are methods for treating hyperammonemia in a patient in need thereof, comprising: a) identifying said patient as having mild renal impairment; and b) administering carglumic acid to said patient at a daily dosage of 100 mg/kg to 250 mg/kg. In some cases, the hyperammonemia is acute hyperammonemia due to NAGS deficiency, MMA, PA, or IVA. In some cases, the hyperammonemia is chronic hyperammonemia due to NAGS deficiency, MMA, PA, or IVA. The daily dosage of carglumic acid for acute hyperammonemia can be about 100 mg/kg to about 250 mg/kg. The daily dosage of carglumic acid can be at least about 100 mg/kg. The daily dosage of carglumic acid can be at most about 250 mg/kg. The daily dosage of carglumic acid can be about 100 mg/kg to about 150 mg/kg, about 100 mg/kg to about 200 mg/kg, about 100 mg/kg to about 250 mg/kg, about 150 mg/kg to about 200 mg/kg, about 150 mg/kg to about 250 mg/kg, or about 200 mg/kg to about 250 mg/kg. The daily dosage of carglumic acid can be about 100 mg/kg, about 150 mg/kg, about 200 mg/kg, or about 250 mg/kg. The daily dosage of carglumic acid can be divided into 2 to 4 doses and/or rounded to the nearest 100 mg (i.e., half of a CARBAGLU® tablet).

Disclosed herein are methods for treating hyperammonemia in a patient in need thereof, comprising: a) identifying said patient as having moderate renal impairment; and b) administering carglumic acid to said patient at a daily dosage of 80 mg/kg to 200 mg/kg. In some cases, the hyperammonemia is acute hyperammonemia due to NAGS deficiency, MMA, PA, or IVA. In some cases, the hyperammonemia is chronic hyperammonemia due to NAGS deficiency, MMA, PA, or IVA. The daily dosage of carglumic acid for acute hyperammonemia can be about 80 mg/kg to about 200 mg/kg. The daily dosage of carglumic acid for acute hyperammonemia can be at least about 80 mg/kg. The daily dosage of carglumic acid for acute hyperammonemia can be at most about 200 mg/kg. The daily dosage of carglumic acid for acute hyperammonemia can be about 80 mg/kg to about 100 mg/kg, about 80 mg/kg to about 120 mg/kg, about 80 mg/kg to about 140 mg/kg, about 80 mg/kg to about 160 mg/kg, about 80 mg/kg to about 180 mg/kg, about 80 mg/kg to about 200 mg/kg, about 100 mg/kg to about 120 mg/kg, about 100 mg/kg to about 140 mg/kg, about 100 mg/kg to about 160 mg/kg, about 100 mg/kg to about 180 mg/kg, about 100 mg/kg to about 200 mg/kg, about 120 mg/kg to about 140 mg/kg, about 120 mg/kg to about 160 mg/kg, about 120 mg/kg to about 180 mg/kg, about 120 mg/kg to about 200 mg/kg, about 140 mg/kg to about 160 mg/kg, about 140 mg/kg to about 180 mg/kg, about 140 mg/kg to about 200 mg/kg, about 160 mg/kg to about 180 mg/kg, about 160 mg/kg to about 200 mg/kg, or about 180 mg/kg to about 200 mg/kg. The daily dosage of carglumic acid for acute hyperammonemia can be about 80 mg/kg, about 100 mg/kg, about 120 mg/kg, about 140 mg/kg, about 160 mg/kg, about 180 mg/kg, or about 200 mg/kg. The daily dosage of carglumic acid can be divided into 2 to 4 doses and/or rounded to the nearest 100 mg (i.e., half of a CARBAGLU® tablet).

Disclosed herein are methods for treating hyperammonemia in a patient in need thereof, comprising: a) identifying said patient as having severe renal impairment; and b) administering carglumic acid to said patient at a daily dosage of 65 mg/kg to 160 mg/kg. In some cases, the hyperammonemia is acute hyperammonemia due to NAGS deficiency, MMA, PA, or IVA. In some cases, the hyperammonemia is chronic hyperammonemia due to NAGS deficiency, MMA, PA, or IVA. The daily dosage of carglumic acid for acute hyperammonemia can be about 65 mg/kg to about 160 mg/kg. The daily dosage of carglumic acid for acute hyperammonemia can be at least about 65 mg/kg. The daily dosage of carglumic acid for acute hyperammonemia can be at most about 160 mg/kg. The daily dosage of carglumic acid for acute hyperammonemia can be about 65 mg/kg to about 80 mg/kg, about 65 mg/kg to about 90 mg/kg, about 65 mg/kg to about 100 mg/kg, about 65 mg/kg to about 110 mg/kg, about 65 mg/kg to about 120 mg/kg, about 65 mg/kg to about 130 mg/kg, about 65 mg/kg to about 140 mg/kg, about 65 mg/kg to about 150 mg/kg, about 65 mg/kg to about 160 mg/kg, about 80 mg/kg to about 90 mg/kg, about 80 mg/kg to about 100 mg/kg, about 80 mg/kg to about 110 mg/kg, about 80 mg/kg to about 120 mg/kg, about 80 mg/kg to about 130 mg/kg, about 80 mg/kg to about 140 mg/kg, about 80 mg/kg to about 150 mg/kg, about 80 mg/kg to about 160 mg/kg, about 90 mg/kg to about 100 mg/kg, about 90 mg/kg to about 110 mg/kg, about 90 mg/kg to about 120 mg/kg, about 90 mg/kg to about 130 mg/kg, about 90 mg/kg to about 140 mg/kg, about 90 mg/kg to about 150 mg/kg, about 90 mg/kg to about 160 mg/kg, about 100 mg/kg to about 110 mg/kg, about 100 mg/kg to about 120 mg/kg, about 100 mg/kg to about 130 mg/kg, about 100 mg/kg to about 140 mg/kg, about 100 mg/kg to about 150 mg/kg, about 100 mg/kg to about 160 mg/kg, about 110 mg/kg to about 120 mg/kg, about 110 mg/kg to about 130 mg/kg, about 110 mg/kg to about 140 mg/kg, about 110 mg/kg to about 150 mg/kg, about 110 mg/kg to about 160 mg/kg, about 120 mg/kg to about 130 mg/kg, about 120 mg/kg to about 140 mg/kg, about 120 mg/kg to about 150 mg/kg, about 120 mg/kg to about 160 mg/kg, about 130 mg/kg to about 140 mg/kg, about 130 mg/kg to about 150 mg/kg, about 130 mg/kg to about 160 mg/kg, about 140 mg/kg to about 150 mg/kg, about 140 mg/kg to about 160 mg/kg, or about 150 mg/kg to about 160 mg/kg. The daily dosage of carglumic acid for acute hyperammonemia can be about 65 mg/kg, about 80 mg/kg, about 90 mg/kg, about 100 mg/kg, about 110 mg/kg, about 120 mg/kg, about 130 mg/kg, about 140 mg/kg, about 150 mg/kg, or about 160 mg/kg. The daily dosage of carglumic acid can be divided into 2 to 4 doses and/or rounded to the nearest 100 mg (i.e., half of a CARBAGLU® tablet).

Disclosed herein are methods for treating hyperammonemia in a patient in need thereof, comprising: a) identifying said patient as having end stage renal impairment; and b) administering carglumic acid to said patient at a daily dosage of 60 mg/kg to 150 mg/kg. In some cases, the hyperammonemia is acute hyperammonemia due to NAGS deficiency, MMA, PA, or IVA. In some cases, the hyperammonemia is chronic hyperammonemia due to NAGS deficiency, MMA, PA, or IVA. The daily dosage of carglumic acid for acute hyperammonemia can be about 60 mg/kg to about 150 mg/kg. The daily dosage of carglumic acid for acute hyperammonemia can be at least about 60 mg/kg. The daily dosage of carglumic acid for acute hyperammonemia can be at most about 150 mg/kg. The daily dosage of carglumic acid for acute hyperammonemia can be about 60 mg/kg to about 70 mg/kg, about 60 mg/kg to about 80 mg/kg, about 60 mg/kg to about 90 mg/kg, about 60 mg/kg to about 100 mg/kg, about 60 mg/kg to about 110 mg/kg, about 60 mg/kg to about 120 mg/kg, about 60 mg/kg to about 130 mg/kg, about 60 mg/kg to about 140 mg/kg, about 60 mg/kg to about 150 mg/kg, about 70 mg/kg to about 80 mg/kg, about 70 mg/kg to about 90 mg/kg, about 70 mg/kg to about 100 mg/kg, about 70 mg/kg to about 110 mg/kg, about 70 mg/kg to about 120 mg/kg, about 70 mg/kg to about 130 mg/kg, about 70 mg/kg to about 140 mg/kg, about 70 mg/kg to about 150 mg/kg, about 80 mg/kg to about 90 mg/kg, about 80 mg/kg to about 100 mg/kg, about 80 mg/kg to about 110 mg/kg, about 80 mg/kg to about 120 mg/kg, about 80 mg/kg to about 130 mg/kg, about 80 mg/kg to about 140 mg/kg, about 80 mg/kg to about 150 mg/kg, about 90 mg/kg to about 100 mg/kg, about 90 mg/kg to about 110 mg/kg, about 90 mg/kg to about 120 mg/kg, about 90 mg/kg to about 130 mg/kg, about 90 mg/kg to about 140 mg/kg, about 90 mg/kg to about 150 mg/kg, about 100 mg/kg to about 110 mg/kg, about 100 mg/kg to about 120 mg/kg, about 100 mg/kg to about 130 mg/kg, about 100 mg/kg to about 140 mg/kg, about 100 mg/kg to about 150 mg/kg, about 110 mg/kg to about 120 mg/kg, about 110 mg/kg to about 130 mg/kg, about 110 mg/kg to about 140 mg/kg, about 110 mg/kg to about 150 mg/kg, about 120 mg/kg to about 130 mg/kg, about 120 mg/kg to about 140 mg/kg, about 120 mg/kg to about 150 mg/kg, about 130 mg/kg to about 140 mg/kg, about 130 mg/kg to about 150 mg/kg, or about 140 mg/kg to about 150 mg/kg. The daily dosage of carglumic acid for acute hyperammonemia can be about 60 mg/kg, about 70 mg/kg, about 80 mg/kg, about 90 mg/kg, about 100 mg/kg, about 110 mg/kg, about 120 mg/kg, about 130 mg/kg, about 140 mg/kg, or about 150 mg/kg. The daily dosage of carglumic acid can be divided into 2 to 4 doses and/or rounded to the nearest 100 mg (i.e., half of a CARBAGLU® tablet).

The daily dosage of carglumic acid for chronic hyperammonemia in patients without renal impairment can be about 10 mg/kg to about 100 mg/kg. The daily dosage of carglumic acid can be at least about 10 mg/kg. The daily dosage of carglumic acid can be at most about 100 mg/kg. The daily dosage of carglumic acid can be about 10 mg/kg to about 20 mg/kg, about 10 mg/kg to about 40 mg/kg, about 10 mg/kg to about 60 mg/kg, about 10 mg/kg to about 80 mg/kg, about 10 mg/kg to about 100 mg/kg, about 20 mg/kg to about 40 mg/kg, about 20 mg/kg to about 60 mg/kg, about 20 mg/kg to about 80 mg/kg, about 20 mg/kg to about 100 mg/kg, about 40 mg/kg to about 60 mg/kg, about 40 mg/kg to about 80 mg/kg, about 40 mg/kg to about 100 mg/kg, about 60 mg/kg to about 80 mg/kg, about 60 mg/kg to about 100 mg/kg, or about 80 mg/kg to about 100 mg/kg. The daily dosage of carglumic acid can be about 10 mg/kg, about 20 mg/kg, about 40 mg/kg, about 60 mg/kg, about 80 mg/kg, or about 100 mg/kg. The daily dosage of carglumic acid can be divided into 2 to 4 doses and/or rounded to the nearest 100 mg (i.e., half of a CARBAGLU® tablet).

### Pharmaceutical Compositions Comprising Carglumic Acid for Use in Treating Hyperammonemia in Patents with Renal Impairment

Patients with Mild Renal Impairment

In certain embodiments, the disclosure is directed to a pharmaceutical composition comprising carglumic acid for use in treating hyperammonemia in a patient in need thereof, comprising: a) identifying said patient as having mild renal impairment; and b) administering said pharmaceutical composition comprising carglumic acid to said patient at a daily dosage of 100 mg/kg to 250 mg/kg. In some cases, the patient has a glomerular filtration rate (GFR) of 60-89 mL/min/1.73 m².

In some cases, the hyperammonemia is acute hyperammonemia due to NAGS deficiency, MMA, PA, or IVA. In some cases, the hyperammonemia is chronic hyperammonemia due to NAGS deficiency, MMA, PA, or IVA.

For patients with mild renal impairment, the daily dosage of carglumic acid for acute hyperammonemia can be about 100 mg/kg to about 250 mg/kg. For patients with mild renal impairment, the daily dosage of carglumic acid for chronic hyperammonemia can be about 10 mg/kg to about 100 mg/kg. The daily dosage of carglumic acid can be at least about 10 mg/kg. For patients with mild renal impairment, the daily dosage of carglumic acid can be at least about 100 mg/kg. The For patients with mild renal impairment, the daily dosage of carglumic acid can be at most about 250 mg/kg. For patients with mild renal impairment, the daily dosage of carglumic acid can be about 100 mg/kg to about 150 mg/kg, about 100 mg/kg to about 200 mg/kg, about 100 mg/kg to about 250 mg/kg, about 150 mg/kg to about 200 mg/kg, about 150 mg/kg to about 250 mg/kg, or about 200 mg/kg to about 250 mg/kg. For patients with mild renal impairment, the daily dosage of carglumic acid can be about 100 mg/kg, about 150 mg/kg, about 200 mg/kg, or about 250 mg/kg. For patients with mild renal impairment, the daily dosage of carglumic acid can be divided into 2 to 4 doses and/or rounded to the nearest 100 mg (i.e., half of a CARBAGLU® tablet).

Patients with Moderate Renal Impairment

In certain embodiments, the disclosure is directed to a pharmaceutical composition comprising carglumic acid for use in treating hyperammonemia in a patient in need thereof, comprising: a) identifying the patient as having moderate renal impairment; and b) administering said pharmaceutical composition comprising carglumic acid to the patient at a daily dosage of 5 mg/kg to 125 mg/kg. In some cases, the patient has a glomerular filtration rate (GFR) of 30-59 mL/min/1.73 m².

In some cases, the hyperammonemia is acute hyperammonemia. In some cases, the hyperammonemia is chronic hyperammonemia In some cases, the hyperammonemia is hyperammonemia due to NAGS deficiency, MMA, PA, or IVA. In some cases, the hyperammonemia is acute hyperammonemia due to NAGS deficiency, MMA, PA, or IVA. In some cases, the hyperammonemia is chronic hyperammonemia due to NAGS deficiency, MMA, PA, or IVA.

In some cases, for patients with moderate renal impairment, the daily dosage of carglumic acid for acute hyperammonemia is about 50 mg/kg to about 125 mg/kg. In some cases, for patients with moderate renal impairment, the daily dosage of carglumic acid for acute hyperammonemia is about 50 mg/kg to about 95 mg/kg. In some cases, for patients with moderate renal impairment, the daily dosage of carglumic acid for acute hyperammonemia is about 50 mg/kg to about 90 mg/kg. In some cases, for patients with moderate renal impairment, the daily dosage of carglumic acid for chronic hyperammonemia is about 10 mg/kg to about 50 mg/kg.

Patients with Severe Renal Impairment

In certain embodiments, the disclosure is directed to a pharmaceutical composition comprising carglumic acid for use in treating hyperammonemia in a patient in need thereof, comprising: a) identifying the patient as having severe renal impairment; and administering said pharmaceutical composition comprising carglumic acid to the patient at a daily dosage of 2 mg/kg to 60 mg/kg. In some cases, the patient has a glomerular filtration rate (GFR) of 15 to 29 mL/min/1.73 m².

In some cases, the hyperammonemia is acute hyperammonemia. In some cases, the hyperammonemia is chronic hyperammonemia In some cases, the hyperammonemia is hyperammonemia due to NAGS deficiency, MMA, PA, or IVA. In some cases, the hyperammonemia is acute hyperammonemia due to NAGS deficiency, MMA, PA, or IVA. In some cases, the hyperammonemia is chronic hyperammonemia due to NAGS deficiency, MMA, PA, or IVA.

In some cases, for patients with severe renal impairment, the daily dosage of carglumic acid for acute hyperammonemia is about 15 mg/kg to about 60 mg/kg. In some cases, for patients with severe renal impairment, the daily dosage of carglumic acid for chronic hyperammonemia is about 2 mg/kg to about 25 mg/kg.

Patients with End Stage Renal Disease

In certain embodiments, the disclosure is directed to a pharmaceutical composition comprising carglumic acid for use in treating hyperammonemia in a patient in need thereof, comprising: identifying the patient as having end stage renal disease; and administering said pharmaceutical composition comprising carglumic acid to the patient at a daily dosage of 2 mg/kg to 60 mg/kg. In some cases, the patient has a glomerular filtration rate (GFR) of less than 15 mL/min/1.73 m².

In some cases, the hyperammonemia is acute hyperammonemia. In some cases, the hyperammonemia is chronic hyperammonemia In some cases, the hyperammonemia is hyperammonemia due to NAGS deficiency, MMA, PA, or IVA. In some cases, the hyperammonemia is acute hyperammonemia due to NAGS deficiency, MMA, PA, or IVA. In some cases, the hyperammonemia is chronic hyperammonemia due to NAGS deficiency, MMA, PA, or IVA.

In some cases, for patients with severe renal impairment, the daily dosage of carglumic acid for acute hyperammonemia is about 15 mg/kg to about 60 mg/kg. In some cases, for patients with severe renal impairment, the daily dosage of carglumic acid for chronic hyperammonemia is about 2 mg/kg to about 25 mg/kg.

### Additional Dose Adjustments

In certain embodiments, the disclosure is directed to a pharmaceutical composition comprising carglumic acid for use in treating hyperammonemia in a patient without renal impairment comprising: a) identifying said patient as not having renal impairment; and b) administering carglumic acid to said patient at a daily dosage of 100 mg/kg to 250 mg/kg. In some cases, the hyperammonemia is acute hyperammonemia due to NAGS deficiency, MMA, PA, or IVA. In some cases, the hyperammonemia is chronic hyperammonemia due to NAGS deficiency, MMA, PA, or IVA. The daily dosage of carglumic acid for acute hyperammonemia can be about 100 mg/kg to about 250 mg/kg. The daily dosage of carglumic acid can be at least about 100 mg/kg. The daily dosage of carglumic acid can be at most about 250 mg/kg. The daily dosage of carglumic acid can be about 100 mg/kg to about 150 mg/kg, about 100 mg/kg to about 200 mg/kg, about 100 mg/kg to about 250 mg/kg, about 150 mg/kg to about 200 mg/kg, about 150 mg/kg to about 250 mg/kg, or about 200 mg/kg to about 250 mg/kg. The daily dosage of carglumic acid can be about 100 mg/kg, about 150 mg/kg, about 200 mg/kg, or about 250 mg/kg. The daily dosage of carglumic acid can be divided into 2 to 4 doses and/or rounded to the nearest 100 mg (i.e., half of a CARBAGLU® tablet).

In certain embodiments, the disclosure is directed to a pharmaceutical composition comprising carglumic acid for use in treating hyperammonemia in a patient having mild renal impairment comprising: a) identifying said patient as having mild renal impairment; and b) administering carglumic acid to said patient at a daily dosage of 100 mg/kg to 250 mg/kg. In some cases, the hyperammonemia is acute hyperammonemia due to NAGS deficiency, MMA, PA, or IVA. In some cases, the hyperammonemia is chronic hyperammonemia due to NAGS deficiency, MMA, PA, or IVA. The daily dosage of carglumic acid for acute hyperammonemia can be about 100 mg/kg to about 250 mg/kg. The daily dosage of carglumic acid can be at least about 100 mg/kg. The daily dosage of carglumic acid can be at most about 250 mg/kg. The daily dosage of carglumic acid can be about 100 mg/kg to about 150 mg/kg, about 100 mg/kg to about 200 mg/kg, about 100 mg/kg to about 250 mg/kg, about 150 mg/kg to about 200 mg/kg, about 150 mg/kg to about 250 mg/kg, or about 200 mg/kg to about 250 mg/kg. The daily dosage of carglumic acid can be about 100 mg/kg, about 150 mg/kg, about 200 mg/kg, or about 250 mg/kg. The daily dosage of carglumic acid can be divided into 2 to 4 doses and/or rounded to the nearest 100 mg (i.e., half of a CARBAGLU® tablet).

In certain embodiments, the disclosure is directed to a pharmaceutical composition comprising carglumic acid for use in treating hyperammonemia in a patient having moderate renal impairment comprising: a) identifying said patient as having moderate renal impairment; and b) administering carglumic acid to said patient at a daily dosage of 80 mg/kg to 200 mg/kg. In some cases, the hyperammonemia is acute hyperammonemia due to NAGS deficiency, MMA, PA, or IVA. In some cases, the hyperammonemia is chronic hyperammonemia due to NAGS deficiency, MMA, PA, or IVA. The daily dosage of carglumic acid for acute hyperammonemia can be about 80 mg/kg to about 200 mg/kg. The daily dosage of carglumic acid for acute hyperammonemia can be at least about 80 mg/kg. The daily dosage of carglumic acid for acute hyperammonemia can be at most about 200 mg/kg. The daily dosage of carglumic acid for acute hyperammonemia can be about 80 mg/kg to about 100 mg/kg, about 80 mg/kg to about 120 mg/kg, about 80 mg/kg to about 140 mg/kg, about 80 mg/kg to about 160 mg/kg, about 80 mg/kg to about 180 mg/kg, about 80 mg/kg to about 200 mg/kg, about 100 mg/kg to about 120 mg/kg, about 100 mg/kg to about 140 mg/kg, about 100 mg/kg to about 160 mg/kg, about 100 mg/kg to about 180 mg/kg, about 100 mg/kg to about 200 mg/kg, about 120 mg/kg to about 140 mg/kg, about 120 mg/kg to about 160 mg/kg, about 120 mg/kg to about 180 mg/kg, about 120 mg/kg to about 200 mg/kg, about 140 mg/kg to about 160 mg/kg, about 140 mg/kg to about 180 mg/kg, about 140 mg/kg to about 200 mg/kg, about 160 mg/kg to about 180 mg/kg, about 160 mg/kg to about 200 mg/kg, or about 180 mg/kg to about 200 mg/kg. The daily dosage of carglumic acid for acute hyperammonemia can be about 80 mg/kg, about 100 mg/kg, about 120 mg/kg, about 140 mg/kg, about 160 mg/kg, about 180 mg/kg, or about 200 mg/kg. The daily dosage of carglumic acid can be divided into 2 to 4 doses and/or rounded to the nearest 100 mg (i.e., half of a CARBAGLU® tablet).

In certain embodiments, the disclosure is directed to a pharmaceutical composition comprising carglumic acid for use in treating hyperammonemia in a patient having severe renal impairment comprising: a) identifying said patient as having severe renal impairment; and b) administering carglumic acid to said patient at a daily dosage of 65 mg/kg to 160 mg/kg. In some cases, the hyperammonemia is acute hyperammonemia due to NAGS deficiency, MMA, PA, or IVA. In some cases, the hyperammonemia is chronic hyperammonemia due to NAGS deficiency, MMA, PA, or IVA. The daily dosage of carglumic acid for acute hyperammonemia can be about 65 mg/kg to about 160 mg/kg. The daily dosage of carglumic acid for acute hyperammonemia can be at least about 65 mg/kg. The daily dosage of carglumic acid for acute hyperammonemia can be at most about 160 mg/kg. The daily dosage of carglumic acid for acute hyperammonemia can be about 65 mg/kg to about 80 mg/kg, about 65 mg/kg to about 90 mg/kg, about 65 mg/kg to about 100 mg/kg, about 65 mg/kg to about 110 mg/kg, about 65 mg/kg to about 120 mg/kg, about 65 mg/kg to about 130 mg/kg, about 65 mg/kg to about 140 mg/kg, about 65 mg/kg to about 150 mg/kg, about 65 mg/kg to about 160 mg/kg, about 80 mg/kg to about 90 mg/kg, about 80 mg/kg to about 100 mg/kg, about 80 mg/kg to about 110 mg/kg, about 80 mg/kg to about 120 mg/kg, about 80 mg/kg to about 130 mg/kg, about 80 mg/kg to about 140 mg/kg, about 80 mg/kg to about 150 mg/kg, about 80 mg/kg to about 160 mg/kg, about 90 mg/kg to about 100 mg/kg, about 90 mg/kg to about 110 mg/kg, about 90 mg/kg to about 120 mg/kg, about 90 mg/kg to about 130 mg/kg, about 90 mg/kg to about 140 mg/kg, about 90 mg/kg to about 150 mg/kg, about 90 mg/kg to about 160 mg/kg, about 100 mg/kg to about 110 mg/kg, about 100 mg/kg to about 120 mg/kg, about 100 mg/kg to about 130 mg/kg, about 100 mg/kg to about 140 mg/kg, about 100 mg/kg to about 150 mg/kg, about 100 mg/kg to about 160 mg/kg, about 110 mg/kg to about 120 mg/kg, about 110 mg/kg to about 130 mg/kg, about 110 mg/kg to about 140 mg/kg, about 110 mg/kg to about 150 mg/kg, about 110 mg/kg to about 160 mg/kg, about 120 mg/kg to about 130 mg/kg, about 120 mg/kg to about 140 mg/kg, about 120 mg/kg to about 150 mg/kg, about 120 mg/kg to about 160 mg/kg, about 130 mg/kg to about 140 mg/kg, about 130 mg/kg to about 150 mg/kg, about 130 mg/kg to about 160 mg/kg, about 140 mg/kg to about 150 mg/kg, about 140 mg/kg to about 160 mg/kg, or about 150 mg/kg to about 160 mg/kg. The daily dosage of carglumic acid for acute hyperammonemia can be about 65 mg/kg, about 80 mg/kg, about 90 mg/kg, about 100 mg/kg, about 110 mg/kg, about 120 mg/kg, about 130 mg/kg, about 140 mg/kg, about 150 mg/kg, or about 160 mg/kg. The daily dosage of carglumic acid can be divided into 2 to 4 doses and/or rounded to the nearest 100 mg (i.e., half of a CARBAGLU® tablet).

In certain embodiments, the disclosure is directed to a pharmaceutical composition comprising carglumic acid for use in treating hyperammonemia in a patient having end stage renal impairment comprising: a) identifying said patient as having end stage renal impairment; and b) administering carglumic acid to said patient at a daily dosage of 60 mg/kg to 150 mg/kg. In some cases, the hyperammonemia is acute hyperammonemia due to NAGS deficiency, MMA, PA, or IVA. In some cases, the hyperammonemia is chronic hyperammonemia due to NAGS deficiency, MMA, PA, or IVA. The daily dosage of carglumic acid for acute hyperammonemia can be about 60 mg/kg to about 150 mg/kg. The daily dosage of carglumic acid for acute hyperammonemia can be at least about 60 mg/kg. The daily dosage of carglumic acid for acute hyperammonemia can be at most about 150 mg/kg. The daily dosage of carglumic acid for acute hyperammonemia can be about 60 mg/kg to about 70 mg/kg, about 60 mg/kg to about 80 mg/kg, about 60 mg/kg to about 90 mg/kg, about 60 mg/kg to about 100 mg/kg, about 60 mg/kg to about 110 mg/kg, about 60 mg/kg to about 120 mg/kg, about 60 mg/kg to about 130 mg/kg, about 60 mg/kg to about 140 mg/kg, about 60 mg/kg to about 150 mg/kg, about 70 mg/kg to about 80 mg/kg, about 70 mg/kg to about 90 mg/kg, about 70 mg/kg to about 100 mg/kg, about 70 mg/kg to about 110 mg/kg, about 70 mg/kg to about 120 mg/kg, about 70 mg/kg to about 130 mg/kg, about 70 mg/kg to about 140 mg/kg, about 70 mg/kg to about 150 mg/kg, about 80 mg/kg to about 90 mg/kg, about 80 mg/kg to about 100 mg/kg, about 80 mg/kg to about 110 mg/kg, about 80 mg/kg to about 120 mg/kg, about 80 mg/kg to about 130 mg/kg, about 80 mg/kg to about 140 mg/kg, about 80 mg/kg to about 150 mg/kg, about 90 mg/kg to about 100 mg/kg, about 90 mg/kg to about 110 mg/kg, about 90 mg/kg to about 120 mg/kg, about 90 mg/kg to about 130 mg/kg, about 90 mg/kg to about 140 mg/kg, about 90 mg/kg to about 150 mg/kg, about 100 mg/kg to about 110 mg/kg, about 100 mg/kg to about 120 mg/kg, about 100 mg/kg to about 130 mg/kg, about 100 mg/kg to about 140 mg/kg, about 100 mg/kg to about 150 mg/kg, about 110 mg/kg to about 120 mg/kg, about 110 mg/kg to about 130 mg/kg, about 110 mg/kg to about 140 mg/kg, about 110 mg/kg to about 150 mg/kg, about 120 mg/kg to about 130 mg/kg, about 120 mg/kg to about 140 mg/kg, about 120 mg/kg to about 150 mg/kg, about 130 mg/kg to about 140 mg/kg, about 130 mg/kg to about 150 mg/kg, or about 140 mg/kg to about 150 mg/kg. The daily dosage of carglumic acid for acute hyperammonemia can be about 60 mg/kg, about 70 mg/kg, about 80 mg/kg, about 90 mg/kg, about 100 mg/kg, about 110 mg/kg, about 120 mg/kg, about 130 mg/kg, about 140 mg/kg, or about 150 mg/kg. The daily dosage of carglumic acid can be divided into 2 to 4 doses and/or rounded to the nearest 100 mg (i.e., half of a CARBAGLU® tablet).

In certain embodiments, the disclosure is directed to the manufacture of a pharmaceutical composition comprising carglumic acid for treating hyperammonemia in a patient without renal impairment comprising: a) identifying said patient as not having renal impairment; and b) administering carglumic acid to said patient at a daily dosage of 100 mg/kg to 250 mg/kg. In some cases, the hyperammonemia is acute hyperammonemia due to NAGS deficiency, MMA, PA, or IVA. In some cases, the hyperammonemia is chronic hyperammonemia due to NAGS deficiency, MMA, PA, or IVA. The daily dosage of carglumic acid for acute hyperammonemia can be about 100 mg/kg to about 250 mg/kg. The daily dosage of carglumic acid can be at least about 100 mg/kg. The daily dosage of carglumic acid can be at most about 250 mg/kg. The daily dosage of carglumic acid can be about 100 mg/kg to about 150 mg/kg, about 100 mg/kg to about 200 mg/kg, about 100 mg/kg to about 250 mg/kg, about 150 mg/kg to about 200 mg/kg, about 150 mg/kg to about 250 mg/kg, or about 200 mg/kg to about 250 mg/kg. The daily dosage of carglumic acid can be about 100 mg/kg, about 150 mg/kg, about 200 mg/kg, or about 250 mg/kg. The daily dosage of carglumic acid can be divided into 2 to 4 doses and/or rounded to the nearest 100 mg (i.e., half of a CARBAGLU® tablet).

In certain embodiments, the disclosure is directed to the manufacture of a pharmaceutical composition comprising carglumic acid for treating hyperammonemia in a patient having mild renal impairment comprising: a) identifying said patient as having mild renal impairment; and b) administering carglumic acid to said patient at a daily dosage of 100 mg/kg to 250 mg/kg. In some cases, the hyperammonemia is acute hyperammonemia due to NAGS deficiency, MMA, PA, or IVA. In some cases, the hyperammonemia is chronic hyperammonemia due to NAGS deficiency, MMA, PA, or IVA. The daily dosage of carglumic acid for acute hyperammonemia can be about 100 mg/kg to about 250 mg/kg. The daily dosage of carglumic acid can be at least about 100 mg/kg. The daily dosage of carglumic acid can be at most about 250 mg/kg. The daily dosage of carglumic acid can be about 100 mg/kg to about 150 mg/kg, about 100 mg/kg to about 200 mg/kg, about 100 mg/kg to about 250 mg/kg, about 150 mg/kg to about 200 mg/kg, about 150 mg/kg to about 250 mg/kg, or about 200 mg/kg to about 250 mg/kg. The daily dosage of carglumic acid can be about 100 mg/kg, about 150 mg/kg, about 200 mg/kg, or about 250 mg/kg. The daily dosage of carglumic acid can be divided into 2 to 4 doses and/or rounded to the nearest 100 mg (i.e., half of a CARBAGLU® tablet).

In certain embodiments, the disclosure is directed to the manufacture of a pharmaceutical composition comprising carglumic acid for treating hyperammonemia in a patient having moderate renal impairment comprising: a) identifying said patient as having moderate renal impairment; and b) administering carglumic acid to said patient at a daily dosage of 80 mg/kg to 200 mg/kg. In some cases, the hyperammonemia is acute hyperammonemia due to NAGS deficiency, MMA, PA, or IVA. In some cases, the hyperammonemia is chronic hyperammonemia due to NAGS deficiency, MMA, PA, or IVA. The daily dosage of carglumic acid for acute hyperammonemia can be about 80 mg/kg to about 200 mg/kg. The daily dosage of carglumic acid for acute hyperammonemia can be at least about 80 mg/kg. The daily dosage of carglumic acid for acute hyperammonemia can be at most about 200 mg/kg. The daily dosage of carglumic acid for acute hyperammonemia can be about 80 mg/kg to about 100 mg/kg, about 80 mg/kg to about 120 mg/kg, about 80 mg/kg to about 140 mg/kg, about 80 mg/kg to about 160 mg/kg, about 80 mg/kg to about 180 mg/kg, about 80 mg/kg to about 200 mg/kg, about 100 mg/kg to about 120 mg/kg, about 100 mg/kg to about 140 mg/kg, about 100 mg/kg to about 160 mg/kg, about 100 mg/kg to about 180 mg/kg, about 100 mg/kg to about 200 mg/kg, about 120 mg/kg to about 140 mg/kg, about 120 mg/kg to about 160 mg/kg, about 120 mg/kg to about 180 mg/kg, about 120 mg/kg to about 200 mg/kg, about 140 mg/kg to about 160 mg/kg, about 140 mg/kg to about 180 mg/kg, about 140 mg/kg to about 200 mg/kg, about 160 mg/kg to about 180 mg/kg, about 160 mg/kg to about 200 mg/kg, or about 180 mg/kg to about 200 mg/kg. The daily dosage of carglumic acid for acute hyperammonemia can be about 80 mg/kg, about 100 mg/kg, about 120 mg/kg, about 140 mg/kg, about 160 mg/kg, about 180 mg/kg, or about 200 mg/kg. The daily dosage of carglumic acid can be divided into 2 to 4 doses and/or rounded to the nearest 100 mg (i.e., half of a CARBAGLU® tablet).

In certain embodiments, the disclosure is directed to the manufacture of pharmaceutical composition comprising carglumic acid for treating hyperammonemia in a patient having severe renal impairment comprising: a) identifying said patient as having severe renal impairment; and b) administering carglumic acid to said patient at a daily dosage of 65 mg/kg to 160 mg/kg. In some cases, the hyperammonemia is acute hyperammonemia due to NAGS deficiency, MMA, PA, or IVA. In some cases, the hyperammonemia is chronic hyperammonemia due to NAGS deficiency, MMA, PA, or IVA. The daily dosage of carglumic acid for acute hyperammonemia can be about 65 mg/kg to about 160 mg/kg. The daily dosage of carglumic acid for acute hyperammonemia can be at least about 65 mg/kg. The daily dosage of carglumic acid for acute hyperammonemia can be at most about 160 mg/kg. The daily dosage of carglumic acid for acute hyperammonemia can be about 65 mg/kg to about 80 mg/kg, about 65 mg/kg to about 90 mg/kg, about 65 mg/kg to about 100 mg/kg, about 65 mg/kg to about 110 mg/kg, about 65 mg/kg to about 120 mg/kg, about 65 mg/kg to about 130 mg/kg, about 65 mg/kg to about 140 mg/kg, about 65 mg/kg to about 150 mg/kg, about 65 mg/kg to about 160 mg/kg, about 80 mg/kg to about 90 mg/kg, about 80 mg/kg to about 100 mg/kg, about 80 mg/kg to about 110 mg/kg, about 80 mg/kg to about 120 mg/kg, about 80 mg/kg to about 130 mg/kg, about 80 mg/kg to about 140 mg/kg, about 80 mg/kg to about 150 mg/kg, about 80 mg/kg to about 160 mg/kg, about 90 mg/kg to about 100 mg/kg, about 90 mg/kg to about 110 mg/kg, about 90 mg/kg to about 120 mg/kg, about 90 mg/kg to about 130 mg/kg, about 90 mg/kg to about 140 mg/kg, about 90 mg/kg to about 150 mg/kg, about 90 mg/kg to about 160 mg/kg, about 100 mg/kg to about 110 mg/kg, about 100 mg/kg to about 120 mg/kg, about 100 mg/kg to about 130 mg/kg, about 100 mg/kg to about 140 mg/kg, about 100 mg/kg to about 150 mg/kg, about 100 mg/kg to about 160 mg/kg, about 110 mg/kg to about 120 mg/kg, about 110 mg/kg to about 130 mg/kg, about 110 mg/kg to about 140 mg/kg, about 110 mg/kg to about 150 mg/kg, about 110 mg/kg to about 160 mg/kg, about 120 mg/kg to about 130 mg/kg, about 120 mg/kg to about 140 mg/kg, about 120 mg/kg to about 150 mg/kg, about 120 mg/kg to about 160 mg/kg, about 130 mg/kg to about 140 mg/kg, about 130 mg/kg to about 150 mg/kg, about 130 mg/kg to about 160 mg/kg, about 140 mg/kg to about 150 mg/kg, about 140 mg/kg to about 160 mg/kg, or about 150 mg/kg to about 160 mg/kg. The daily dosage of carglumic acid for acute hyperammonemia can be about 65 mg/kg, about 80 mg/kg, about 90 mg/kg, about 100 mg/kg, about 110 mg/kg, about 120 mg/kg, about 130 mg/kg, about 140 mg/kg, about 150 mg/kg, or about 160 mg/kg. The daily dosage of carglumic acid can be divided into 2 to 4 doses and/or rounded to the nearest 100 mg (i.e., half of a CARBAGLU® tablet).

In certain embodiments, the disclosure is directed to the manufacture of a pharmaceutical composition comprising carglumic acid for treating hyperammonemia in a patient having end stage renal impairment comprising: a) identifying said patient as having end stage renal impairment; and b) administering carglumic acid to said patient at a daily dosage of 60 mg/kg to 150 mg/kg. In some cases, the hyperammonemia is acute hyperammonemia due to NAGS deficiency, MMA, PA, or IVA. In some cases, the hyperammonemia is chronic hyperammonemia due to NAGS deficiency, MMA, PA, or IVA. The daily dosage of carglumic acid for acute hyperammonemia can be about 60 mg/kg to about 150 mg/kg. The daily dosage of carglumic acid for acute hyperammonemia can be at least about 60 mg/kg. The daily dosage of carglumic acid for acute hyperammonemia can be at most about 150 mg/kg. The daily dosage of carglumic acid for acute hyperammonemia can be about 60 mg/kg to about 70 mg/kg, about 60 mg/kg to about 80 mg/kg, about 60 mg/kg to about 90 mg/kg, about 60 mg/kg to about 100 mg/kg, about 60 mg/kg to about 110 mg/kg, about 60 mg/kg to about 120 mg/kg, about 60 mg/kg to about 130 mg/kg, about 60 mg/kg to about 140 mg/kg, about 60 mg/kg to about 150 mg/kg, about 70 mg/kg to about 80 mg/kg, about 70 mg/kg to about 90 mg/kg, about 70 mg/kg to about 100 mg/kg, about 70 mg/kg to about 110 mg/kg, about 70 mg/kg to about 120 mg/kg, about 70 mg/kg to about 130 mg/kg, about 70 mg/kg to about 140 mg/kg, about 70 mg/kg to about 150 mg/kg, about 80 mg/kg to about 90 mg/kg, about 80 mg/kg to about 100 mg/kg, about 80 mg/kg to about 110 mg/kg, about 80 mg/kg to about 120 mg/kg, about 80 mg/kg to about 130 mg/kg, about 80 mg/kg to about 140 mg/kg, about 80 mg/kg to about 150 mg/kg, about 90 mg/kg to about 100 mg/kg, about 90 mg/kg to about 110 mg/kg, about 90 mg/kg to about 120 mg/kg, about 90 mg/kg to about 130 mg/kg, about 90 mg/kg to about 140 mg/kg, about 90 mg/kg to about 150 mg/kg, about 100 mg/kg to about 110 mg/kg, about 100 mg/kg to about 120 mg/kg, about 100 mg/kg to about 130 mg/kg, about 100 mg/kg to about 140 mg/kg, about 100 mg/kg to about 150 mg/kg, about 110 mg/kg to about 120 mg/kg, about 110 mg/kg to about 130 mg/kg, about 110 mg/kg to about 140 mg/kg, about 110 mg/kg to about 150 mg/kg, about 120 mg/kg to about 130 mg/kg, about 120 mg/kg to about 140 mg/kg, about 120 mg/kg to about 150 mg/kg, about 130 mg/kg to about 140 mg/kg, about 130 mg/kg to about 150 mg/kg, or about 140 mg/kg to about 150 mg/kg. The daily dosage of carglumic acid for acute hyperammonemia can be about 60 mg/kg, about 70 mg/kg, about 80 mg/kg, about 90 mg/kg, about 100 mg/kg, about 110 mg/kg, about 120 mg/kg, about 130 mg/kg, about 140 mg/kg, or about 150 mg/kg. The daily dosage of carglumic acid can be divided into 2 to 4 doses and/or rounded to the nearest 100 mg (i.e., half of a CARBAGLU® tablet).

### Administration and Treatment

In certain embodiments, carglumic acid can be administered by any suitable means. In other embodiments, carglumic acid can be delivered parenterally. In further embodiments, carglumic acid can be delivered intravenously. In some embodiments, carglumic acid can be administered orally.

In particular embodiments, carglumic acid is delivered as a parenteral formulation as described in WO/2018/095848. In still other embodiments, carglumic acid can be administered as a dispersible tablet. For example, and without limitation, carglumic acid can be dispersed in water by mixing 200 mg of carglumic acid (i.e., a CARBAGLU® tablet) in a minimum of 2.5 mL of water. A dispersed carglumic acid solution can have a concentration of about 80 mg/mL. In other embodiments, carglumic acid can be administered as a soluble tablet.

The carglumic acid solution can be administered orally to pediatric and adult patients. The carglumic acid solution can be taken immediately before meals or feedings. The carglumic acid solution can be administered via a nasogastric tube to pediatric and adult patients. The carglumic acid solution can be administered orally via an oral syringe in pediatric patients.

Carglumic acid can be used in combination with one or more pharmaceutic agents for treating hyperammonemia, including intravenous arginine, sodium phenylbutyrate, and sodium benzoate. Carglumic acid can be used in combination with dialysis, such as hemodialysis and/or hemofiltration.

Carglumic acid can be used in combination with complete protein restriction for no longer than 12 to 48 hours during an acute hyperammonemic episode while maximizing caloric supplementation to reverse catabolism.

### EXAMPLES

### Example 1—Safety Tests for Carglumic Acid

A total of 80 patients were treated with CARBAGLU® (carglumic acid) in patients with hyperammonemia (23 with NAGS deficiency, 27 with PA, 24 with MMA, 5 with IVA, and 1 with succinyl-Patients with NAGS deficiency were <30 days old to 13 years old at initiation of treatment; the mean (SD) age was 2 (4) years. Sixty-one percent (61%) of patients were male and 39% of patients were female.

In patients with PA, MMA or IVA (48% male and 52% female patients), 57% of decompensation episodes occurred during the first 4 weeks after birth (neonates), and 43% of decompensation episodes occurred beyond the neonatal period. The median age at the start of the decompensation episode was 12 days.

The adverse reactions occurring in 2 or more patients (>3%) with NAGS deficiency, PA, MMA, or IVA treated with carglumic acid in the retrospective studies are summarized in Table 3.

TABLE 3
Adverse Reactions Reported in ≥2 Patients (≥3%)
with 142 NAGS deficiency, PA, MMA, or IVA Treated with
CARBAGLU ® (carglumic acid) in the Retrospective Studies

| | Number of Patients (N) (%) |
|---|---|
| Adverse Reaction | Total Population = 80 |
| Pyrexia | 7 (9%) |
| Vomiting | 6 (8%) |
| Diarrhea | 5 (5%) |
| Abdominal pain | 4 (5%) |
| Tonsillitis | 4 (5%) |
| Anemia | 4 (5%) |
| Ear infection | 3 (4%) |
| Infection | 3 (4%) |
| Nasopharyngitis | 3 (4%) |
| Headache | 3 (4%) |
| Hyperhidrosis | 2 (3%) |
| Influenza | 2 (3%) |
| Pneumonia | 2 (3%) |
| Hemoglobin decreased | 2 (3%) |
| Weight decreased | 2 (3%) |
| Anorexia | 2 (3%) |
| Somnolence | 2 (3%) |
| Rash | 2 (3%) |
| Jaundice | 2 (3%) |
| Transaminase increased | 2 (3%) |
| Nervous system disorder | 2 (3%) |
| Dysgeusia | 2 (3%) |

### Example 2—Pediatric Use of Carglumic Acid

The efficacy of carglumic acid for the treatment of hyperammonemia in patients with NAGS deficiency from birth to adulthood was evaluated in a retrospective review of the clinical course of 23 NAGS deficiency patients who all began carglumic acid treatment during infancy or childhood. There are no apparent differences in clinical response between adults and pediatric NAGS deficiency patients treated with carglumic acid.

The efficacy of carglumic acid for the treatment of acute hyperammonemia was evaluated in 51 patients (27 patients with PA and 24 patients with MMA) treated with carglumic acid. All patients were under 16 years of age. There are no apparent differences in plasma ammonia level reduction between pediatric and adult patients with PA or MMA.

### Example 3—Pharmacokinetics Study of Carglumic Acid

The pharmacokinetics of carglumic acid were studied in healthy subjects using both radiolabeled and non-radiolabeled CARBAGLU® (carglumic acid) and by monitoring of plasma levels in patients.

Absorption

The Tₘₐₓ of carglumic acid ranges from 2 to 4 hours with a median of 3 hours. Absolute bioavailability is 9.9%.

Distribution

The apparent volume of distribution determined after oral administration ranges from 1616-5797 L, with a median of 2657 L, which corresponds to 35.9 L/kg. The median volume of distribution determined after IV administration is 10.2 L/kg. The parent product is not bound to plasma.

Elimination

The product was predominantly excreted by the kidneys as unchanged product: only a minor part was metabolized. A proportion of carglumic acid was metabolized by the intestinal bacterial flora. The likely end product of carglumic acid metabolism was carbon dioxide, eliminated through the lungs. In vitro hepatic metabolism has not been observed.

After oral administration, the initial half-life of carglumic acid was approximately 5.6 hours, and the terminal half-life was approximately 22 hours. The median apparent total clearance was 5.7 L/min (3.0 to 9.7 L/min), the median renal clearance was 290 mL/min (204 to 445 mL/min), and the 24-hour urinary excretion was 4.5% of the dose (3.5% to 7.5%) is excreted in the urine over 24 hours. Following oral administration of a single radiolabeled oral carglumic acid dose of 100 mg/kg of body weight, 9% of the dose was excreted unchanged in the urine and up to 60% of the dose was recovered unchanged in the feces.

After IV administration, the median terminal half-life was approximately 22 hours (7 to 36 h). The median total clearance was 0.354 L/min (0.241 to 0.502 L/min), and 75% of the dose (59 to 86%) was excreted in the urine as parent compound over 24 hours. The median renal clearance was 265 mL/min (183 to 467 mL/min), and inter-individual variability (CV %) is 17%.

Drug Interaction Studies

Based on in vitro studies, carglumic acid was not an inducer of CYP1A1/2, CYP2B6, CYP2C, and CYP3A4/5 enzymes, and not an inhibitor of CYP1A2, CYP2A6, CYP2B6, CYP2C8, CYP2C9, CYP2C19, CYP2D6, CYP2E1, and CYP3A4/5 enzymes.

In vitro drug interaction studies were performed with human transporters. Carglumic acid was not an inhibitor of human BSEP, BCRP, MDR1 efflux (ABC) transporters or of human MATE1, MATE2-K, OAT1, OAT3, OATP1B1, OATP1B3, OCT1, and OCT2 uptake transporters. Carglumic acid was a substrate of the human OAT1 transporter which could contribute to an active excretion in the kidneys. Potential interactions with inhibitors of the OAT1 transporter were considered clinically irrelevant.

### Example 4—Efficacy of Carglumic Acid in the Treatment of Hyperammonemia Due to NAGS Deficiency

The efficacy of carglumic acid in the treatment of hyperammonemia due to NAGS deficiency was evaluated in a retrospective review of the clinical course of 23 NAGS deficiency patients who received CARBAGLU® (carglumic acid) treatment for a median of 7.9 years (range 0.6 to 20.8 years). Treatment with carglumic acid was divided in two regimens. For acute treatment, patients received a total daily dose of 100 mg/kg/day to 250 mg/kg/per day primarily administered in 2 to 4 divided doses for the first few days of treatment. For maintenance treatment, the dosage was reduced over time based on plasma ammonia level and clinical responses. The demographic characteristics of the patient population are shown in Table 4.

TABLE 4
Baseline Characteristics of the 23 NAGS Deficiency Patients

| | | Patients | |
|---|---|---|---|
| | | N = 23 | |
| Gender | Male | 14 | (61%) |
| | Female | 9 | (39%) |
| Age at initiation of CARBAGLU | Mean (SD) | 2 | (4) |
| therapy (years) | Min-Max | 0-13 | |
| Age groups at initiation of | <30 days | 9 | (39%) |
| CARBAGLU therapy | >30 days-11 months | 9 | (39%) |
| | ≥1-13 years | 5 | (22%) |
| NAGS gene mutations by DNA | Homozygous | 14 | (61%) |
| testing | Heterozygous | 4 | (17%) |
| | Not available | 5 | (22%) |
| Patients' current treatment status | On-going | 18 | (78%) |
| | Discontinued | 5 | (22%) |

Short-term efficacy was evaluated using mean and median change in plasma ammonia levels from baseline to days 1 to 3. Persistence of efficacy was evaluated using long-term mean and median change in plasma ammonia level. Table 5 summarizes the plasma ammonia levels at baseline, days 1 to 3 post-carglumic acid treatment, and long-term carglumic acid treatment (mean 8 years) for 13 evaluable patients. Of the 23 NAGS deficiency patients who received treatment with carglumic acid, a subset of 13 patients who had both well documented plasma ammonia levels prior to carglumic acid treatment and after long-term treatment with carglumic acid were selected for analysis.

All 13 patients had abnormal plasma ammonia levels at baseline. The overall mean baseline plasma ammonia level was 271 micromol/L. By day 3, normal plasma ammonia levels were attained in patients for whom data were available. Long-term efficacy was measured using the last reported plasma ammonia level for each of the 13 patients analyzed (median length of treatment was 6 years; range 1 to 16 years). The mean and median plasma ammonia levels were 23 micromol/L and 24 micromol/L, respectively, after a mean treatment duration of 8 years (Table 5).

TABLE 5
Plasma Ammonia Levels at Baseline and
After Treatment with Carglumic Acid

| | Statistics | Ammonia ** |
|---|---|---|
| Timepoint | (N = 13*) | (micromole/L) |
| Baseline | N | 13 |
| (prior to first treatment | Mean (SD) | 271 (359) |
| with CARBAGLU) | Median | 157 |
| | Range | 72-1428 |
| | Missing Data | 0 |
| Day 1 | N | 10 |
| | Mean (SD) | 181 (358) |
| | Median | 65 |
| | Range | 25-1190 |
| | Missing Data | 3 |
| Day 2 | N | 8 |
| | Mean (SD) | 69 (78) |
| | Median | 44 |
| | Range | 11-255 |
| | Missing Data | 5 |
| Day 3 | N | 5 |
| | Mean (SD) | 27 (11) |
| | Median | 25 |
| | Range | 12-42 |
| | Missing Data | 8 |
| Long-term | N | 13 |
| Mean: 8 years | Mean (SD) | 23 (7) |
| Median: 6 years | Median | 24 |
| 1 to 16 years | Range | 9-34 |
| (last available value on | Missing Data | 0 |
| CARBAGLU treatment) | | |

*13/23 patients with complete short-term and long-term plasma ammonia documentation
** Mean plasma ammonia normal range: 5 to 50 micromol/L

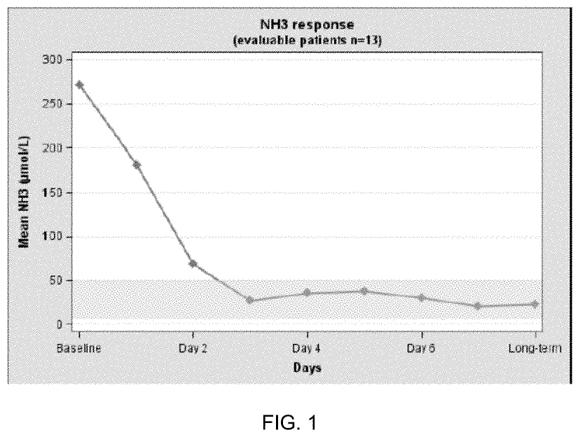

The mean plasma ammonia level at baseline and the decline that is observed after treatment with carglumic acid in 13 evaluable patients with NAGS deficiency is illustrated in FIG. 1.

### Example 5—Efficacy of Carglumic Acid in the Treatment of Acute Hyperammonemia Due to PA and MMA

The efficacy of carglumic acid in the treatment of hyperammonemia due to PA, MMA, and IVA during decompensation episodes was evaluated in a retrospective review of patients treated with CARBAGLU® (carglumic acid).

Patients were given carglumic acid with or without ammonia scavengers or ammonia scavengers (NH₃ Scav) alone. Plasma ammonia (NH₃) levels were measured up to a maximum of 15 days from the start of treatment. Patients received 100 to 250 mg/kg of carglumic acid per day administered in 2 to 4 divided doses; the mean (SD) daily dose of carglumic acid during the first 24 hours of treatment was 158.8 (111.4) mg/kg. Carglumic acid was used to treat hyperammonemia in addition to standard of care treatment of the acute decompensation episode meant to reverse catabolism and promote anabolism. Concomitant standard of care therapies included carnitine, arginine, hydroxocobalamin, biotin, thiamine, riboflavin and glucose.

The 3 separate treatment groups (1—carglumic acid alone, 2—NH₃ Scav alone, and 3—carglumic acid with NH₃ Scav) were compared to examine the isolated effect of carglumic acid without concomitant ammonia-reducing treatments including extracorporeal detoxification (hemodialysis [HD], hemofiltration [HF] or peritoneal dialysis [PD]) and ammonia scavengers.

The mean baseline plasma NH₃ levels (SD) for carglumic acid alone, NH₃ Scav alone, and carglumic acid with NH₃ Scav treatment groups were 257.0 (268.4), 226.8 (287.8), and 345.6 (274.7), respectively. The median duration of treatment was 4.0 days in the carglumic acid alone and NH₃ Scav alone groups, and 5.0 days in the carglumic acid with NH₃ Scav group. In all groups, the delay between start of episode and start of treatment was 1 day.

The number of patients (78) and episodes (98) analyzed per treatment group included: carglumic acid alone group: 34 patients who experienced 38 episodes; NH₃ Scav alone group: 22 patients who experienced 33 episodes; and carglumic acid with NH₃ Scav group: 27 patients who experienced 27 episodes.

The baseline demographics of this patient population are shown in Table 6.

TABLE 6
Demographics 411 (N = 98 Episodes)

| | | | CARBAGLU |
|---|---|---|---|
| | | NH₃ | with NH₃ |
| | | Scav- | Scav- |
| | CARBAGLU | engers | engers |
| | Episodes | Episodes | Episodes |
| Description | (N = 38) | (N = 33) | (N = 27) |

Diagnosis

| | | | | |
|---|---|---|---|---|
| PA | n (%) of episodes | 19 (50.0) | 27 (81.8) | 14 (51.9) |
| MMA | n (%) of episodes | 15 (39.5) | 5 (15.2) | 12 (44.4) |
| IVA | n (%) of episodes | 4 (10.5) | 1 (3.0) | 1 (3.7) |

Gender

| | | | | |
|---|---|---|---|---|
| Female | n (%) of episodes | 16 (42.1) | 17 (51.5) | 15 (55.6) |
| Male | n (%) of episodes | 22 (57.9) | 16 (48.5) | 12 (44.4) |

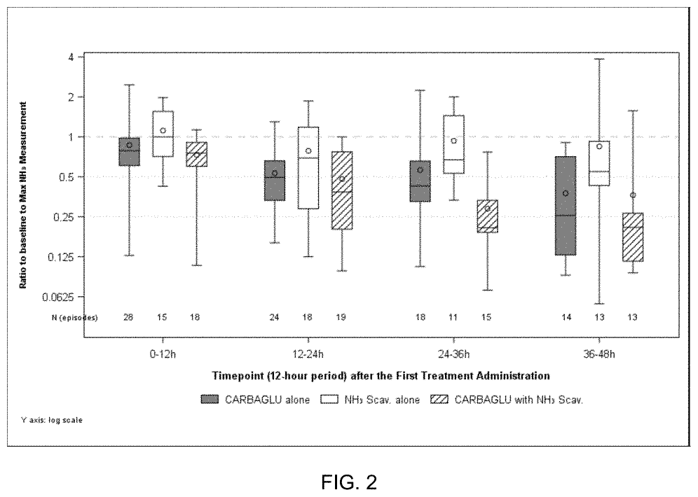

During the 12-hour periods from treatment initiation to 48 hours, the mean reduction in plasma NH₃ levels from baseline in the carglumic acid alone group (36-48 hours: −63%) was greater than in the NH₃ Scav group (36-48 hours: −15%) [p=0.0348] (see FIG. 2).

Overall, the number of clinical findings, including clinical, laboratory, neurological, psychiatric, and respiratory findings and feeding difficulties, from baseline to endpoint decreased in all treatment groups. The median change in the number of findings from baseline to endpoint was −4.0 in the carglumic acid alone group and −3.0 in both the carglumic acid with NH₃ Scav and the NH₃ Scav alone groups.

The normalization of the neurological findings occurred for 50%, 36%, and 45% of episodes in the carglumic acid alone group, the NH₃ Scav alone group, and the carglumic acid with NH₃ Scav group, respectively.

### Example 6—Mean Percent Reduction of Plasma NH₃ in Propensity Matching Score Population

An analysis of plasma NH₃ reduction in patients comparing carglumic acid alone to NH₃ Scav alone in patients matched for baseline plasma NH₃ levels and neonate/non-neonate co-variants was conducted using propensity matching score (PMS Population). Episodes treated with extracorporeal detoxification (HD, HF, PD) during the timeframe of the analysis were excluded.

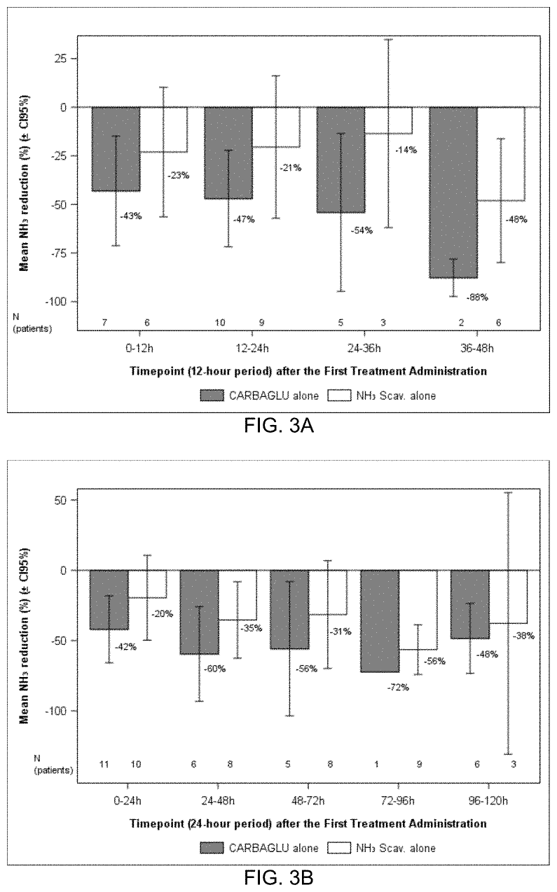

There were 12 patients in each group, and NH₃ levels were measured in 12- and 24-hour periods over 120 hours. A consistent and meaningful reduction of mean plasma NH₃ levels from baseline with carglumic acid alone compared with NH₃ Scav alone was found when all available variables were equal (see FIG. 3).

### Example 7—Analysis of Effect of Carglumic Acid on Rate of Reduction of Plasma NH₃ Over 36 Hours

Analyses of the rate of plasma ammonia reduction in the critical first 36 hours after treatment initiation with carglumic acid were conducted in population excluding patients with extracorporeal detoxification. In the carglumic acid alone group and the carglumic acid with NH₃ Scav group, plasma NH₃ levels were reduced statistically significantly faster than in the NH₃ Scav alone group in the two populations analyzed.

The two populations analyzed included: 1) Population with baseline plasma NH₃ levels over 100 micromol/L in non-neonate or 150 micromol/L in neonate; 2) Propensity Matching Score (PMS) Population.

Results in Population with Baseline Plasma NH₃ Levels Over 100 Micromol/L in Non-Neonate or 150 Micromol/L in Neonate

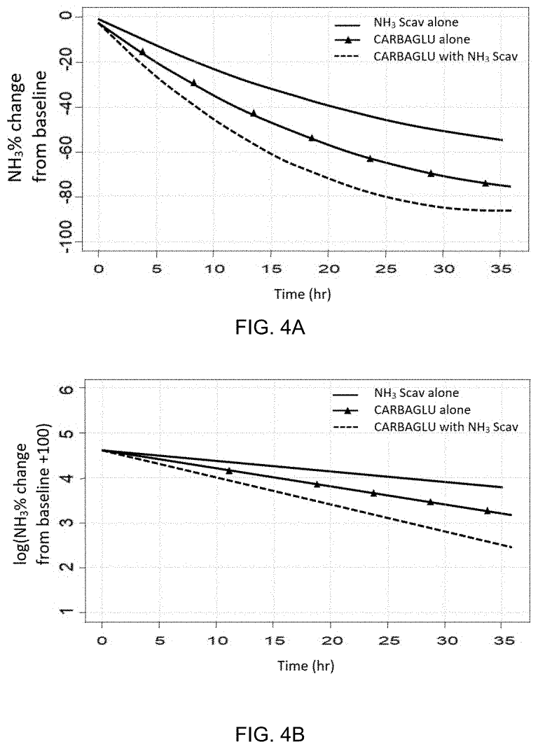

A faster decrease of plasma NH₃ levels was observed in patients treated with carglumic acid alone (median time to achieve 50% reduction was 18 hours post dose) and carglumic acid with NH₃ Scav (median time to achieve 50% reduction was 12 hours post dose) versus NH₃ Scav alone (median time to achieve 50% reduction was 34 hours post dose). These rates of decrease were confirmed to be statistically significant. FIGS. 4A and 4B displays the rate of reduction of plasma NH₃ comparing the 3 treatment groups in patients with baseline plasma NH₃ levels over 100 micromol/L in non-neonate or 150 micromol/L in neonate.

Results in Propensity Matching Score (PMS) Population

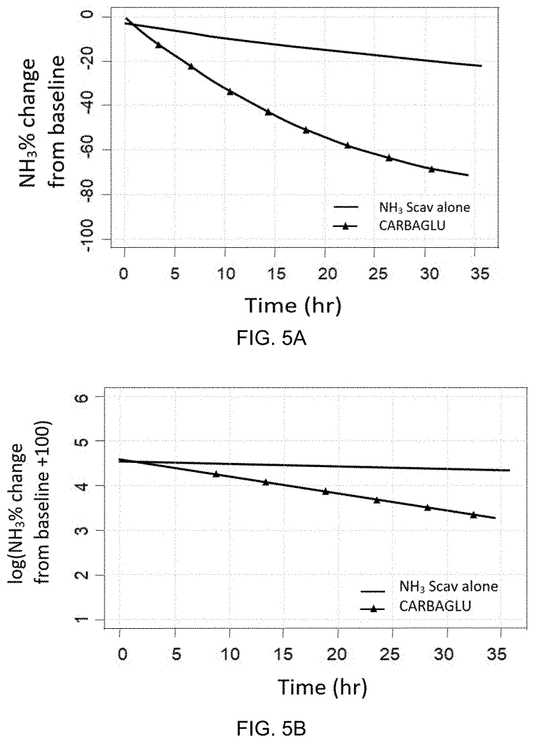

A faster decrease of plasma NH₃ levels was observed in patients treated with carglumic acid alone (61% median reduction at 24 hours post dose) compared to the NH₃ Scav alone (19% median reduction at 24 hours post dose). These rates of decrease were confirmed to be statistically significant. FIG. 5 displays the rate of reduction of plasma NH₃ comparing treatment with NH₃ Scav alone or carglumic acid alone in the PMS Population.

### Example 8—Bibliographic Review of Case Reports in PA, MMA, and IVA Patients Treated with Carglumic Acid Alone for Acute Hyperammonemia

To support the efficacy of carglumic acid in this rare population, a bibliographic review was conducted. A total of 22 patients who experienced 29 episodes of acute metabolic decompensation and hyperammonemia were identified for analysis: 16 patients had PA, 5 patients had MMA, and 1 patient had IVA. The gender ratio was 12 males to 7 females; gender was unknown in 3 cases. The median age was 6 days (range: 1 day to 4 years). CARBAGLU® (carglumic acid) was given to reduce plasma ammonia as adjunctive therapy to standard of care of acute metabolic decompensation. The management of the acute decompensation included IV glucose, insulin, carnitine, antibiotics, intralipids, arginine, hydroxocobalamin, biotin, or forced diuresis. Patients were treated with carglumic acid with or without NH₃ scavengers.

A total of 13 episodes (corresponding to 9 patients) were treated with carglumic acid alone as an adjunctive therapy to standard care, without NH₃ Scav or extracorporeal detoxification (HD, HF, PD) as described in Table 7. These episodes allow for assessment of the isolated effect of carglumic acid. During the first 24 hours of treatment, the mean (SD) dose of carglumic acid was 126 (52) mg/kg and the median dose of carglumic acid was 104 mg/kg (range: 68 mg/kg to 240 mg/kg). In most cases, a marked reduction in plasma NH₃ levels was observed within the first 6 hours of carglumic acid treatment, and the values were normalized within 30 hours of treatment. All patients recovered from the hyperammonemia and acute decompensation.

TABLE 7
Summary of Plasma Ammonia Levels - Episodes Treated with Carglumic
Acid Alone (without NH₃ Scav or Extracorporeal Detoxification)

| | Reference | | Time-Points | |
|---|---|---|---|---|
| | unit Episode | Baseline¹ | First 24 hrs² | First 48 hrs³ |
| All - NH₃ level | N | 13 | 12 | 13 |
| (micromol/L) | Mean (SD) | 349.7 (262.1) | 94.6 (47.6) | 80.5 (43.4) |
| | Median | 264.8 | 100.0 | 66.0 |
| | Q1-Q3 | 217.0-333.5 | 50.0-110.5 | 50.0-100.0 |
| | Min-Max | 112.0-1089.0 | 43.3-187.9 | 32.0-187.9 |
| All - NH₃ level | N | 5 | 5 | 5 |
| (micromol/L) | Mean (SD) | 526.3 (365.4) | 78.5 (29.6) | 78.5 (29.6) |
| | Median | 487.0 | 100.0 | 100.0 |
| | Q1-Q3 | 333.5-610.0 | 50.0-100.0 | 50.0-100.0 |
| | Min-Max | 112.0-1089.0 | 42.3-100.0 | 42.3-100.0 |
| Non- | N | 8 | 7 | 8 |
| Neonates- | Mean (SD) | 239.3 (72.6) | 106.1 (56.6) | 81.8 (52.2) |
| NH₃ level | Median | 243.5 | 100.0 | 58.0 |
| (micromol/L) | Q1-Q3 | 190.7-295.4 | 50.0-170.0 | 48.8-110.5 |
| | Min-Max | 122.0-333.0 | 47.6-187.9 | 32.0-187.9 |

¹Baseline: The last plasma NH₃ value before initiation of carglumic acid.
²24 hrs: The last plasma NH₃ value measured in the 24 hrs after initiation of carglumic acid.
³48 hrs: The last plasma NH₃ value measured in the 48 hrs after initiation of carglumic acid.

### Example 9—Pharmacokinetics Study of Carglumic Acid

A pharmacokinetics (PK) study compared oral to IV CARBAGLU® (carglumic acid) in 36 healthy volunteers (HV). The IV formulation of carglumic acid was administered at single ascending doses at different infusion durations to evaluate the absolute bioavailability and to assess the fraction of the renally excreted dose. The mean absolute bioavailability was 9.87% and the mean fraction of the dose excreted in the urine after IV administration was 90% while after oral administration was 7.5%.

A population PK model was developed using PK data collected from the PK study mentioned above. The data was collected after variable long treatment periods lasting more than a year in some cases, with different doses, and with different dosage regimens in both pediatric and adult volunteers (N=16).

The sparse drug concentrations (24 PK samples) were fitted using a two-compartment model with zero order absorption rate, which was also used to describe the PK time course in the PK study. Descriptive statistics including the age and weight of the study population are shown in Table 8.

TABLE 8
Descriptive statistics of the age and weight of the study population

| Variable | N | Mean | Median | Min | Max |
|---|---|---|---|---|---|
| Age(year) | 16 | 7.56 | 6.5 | 0.005 | 33 |
| Weight(kg) | 16 | 26.74 | 23.45 | 3.27 | 63 |

To account for growth effects, body weight (WT) was included in the model, using an allometric scaling component on clearance (CL) and volume of distribution (V), with power coefficients of 0.75 and 1.0, respectively (Anderson, B. J. & Holford, N. H. Mechanistic basis of using body size and maturation to predict clearance in humans. Drug Metab. Pharmacokinet. 24, 25-36 (2009)). Furthermore, considering that the age of the patients varied between 2 days to 33 years, an age-related effect on the clearance efficiency was added to the model to account for the level of organ maturation in the neonate subjects (Tod M, Lokiec F, Bidault R, De Bony F, Petitjean O, Aujard Y. Pharmacokinetics of oral acyclovir in neonates and in infants: a population analysis. Antimicrob Agents Chemother. 2001; 45(1):150-7).

The final models for Clearance and Volume were:

$$C L = C {L}_{t} \times {[ \frac{W T}{70} ]}^{0.75} \times \frac{A g {e}^{g}}{A g {e}^{g} + TM {50}^{g}} \\ V = {V}_{t} \times {[ \frac{W T}{70} ]}^{1}$$

Wherein CL is the value of the clearance in a patient, CLₜ is the typical value of the clearance in adult subjects, WT is the body weight, TM50 is the maturation half time, g is the Hill coefficient and Vₜ is the typical value of the volume in adult subjects. The CLₜ and the Vₜ values were fixed to the values estimated in HVs: 0.178 and 1820 respectively.

### Example 10—Modeling of Dosage Adjustments for Patients with Renal Impairment

The simulations for patients with renal impairment were conducted using the PK model. The classification of renal impairment was based on the FDA Guidance for Industry Pharmacokinetics in Patients with Impaired Renal Function—Study Design, Data Analysis, and Impact on Dosing and Labeling, March 2010. Three simulation scenarios were considered assuming that in a subject with normal renal function 90% of the available dose is excreted by the kidney (CL_R=90% of the total CL) and 10% by other routes (CL_NR=10% of the total CL).

1) No renal impairment (Control CLcr≥90 mL/min):

CL=RefCL (the reference clearance);

2) Mild renal impairment (CLcr 60-89 mL/min) the CL_R is reduced by about 25%:

CL=CL_NR (10% of RefCL)+CL_R (75% of RefCL);

3) Moderate renal impairment (CLcr=30-59 mL/min) the CL_R is reduced by about 50%:

CL=CL_NR (10% of RefCL)+CL_R (45% of RefCL);

4) Severe renal impairment (CLcr=15-29 mL/min) the CL_R is reduced by about 80%:

CL=CL_NR (10% of RefCL)+CL_R (18% of RefCL).

5) End stage renal disease (CLcr<15 mL/min not on dialysis/requiring dialysis) the CL_R is reduced by about 90%:

CL=CL_NR (10% of RefCL)+CL_R (10% of RefCL).

Due to the age and weight dependency of the values of CL and Volume in the model, the following simulation scenarios were considered (Table 9):

TABLE 9
Simulation scenarios evaluated

| | | |
|---|---|---|
| Scenario | Age | Weight (kg) |

| | | | |
|---|---|---|---|
| 1 | 20 | days | 3.5 |
| 2 | 1 | year | 9.5 |
| 3 | 15 | years | 50.0 |

A Monte Carlo simulation approach was used in the assessment of the simulation outcomes by resampling PK data in 500 subjects. The results of the simulations are presented as median predicted values.

All data preparation, summary statistics and graphical display presentation was performed using R (3.2.5 version) and SAS (version 9.4). The modeling and simulation analysis was conducted using NONMEM (version 7.4.1).

The simulations were conducted using a sequential approach: In the first step carglumic acid exposure was estimated in subjects with normal, mild, moderate, severe, and end stage renal disease using the dosing regimen of 100 mg/kg/day and 250 mg/kg/day; In the second step a dose correction was estimated to provide similar exposure in the subjects with mild, moderate, severe, and end stage renal disease to the exposure estimated in the subjects with normal renal functions.

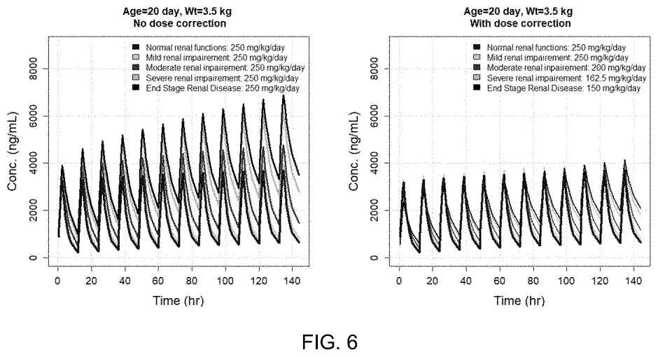

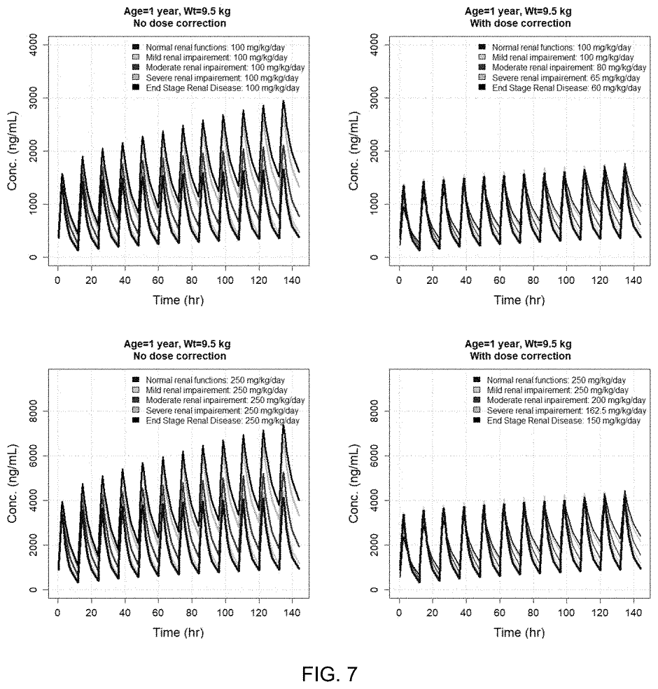

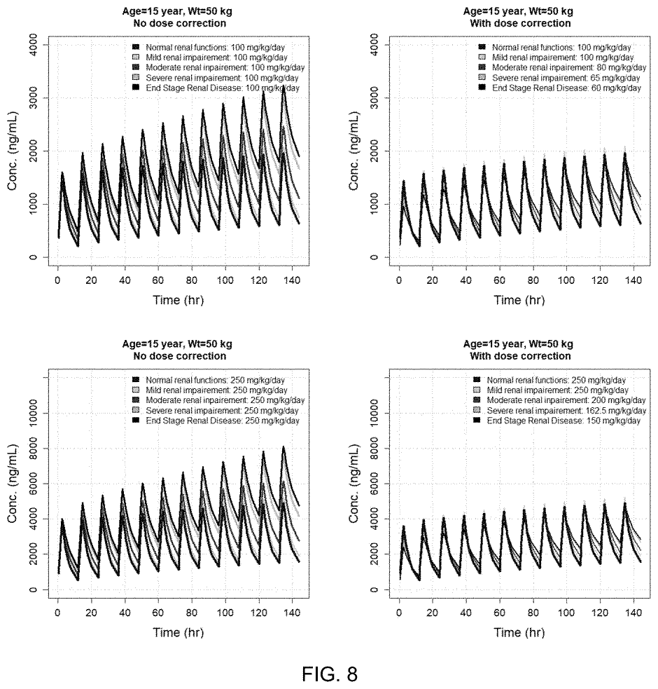

FIGS. 6, 7, and 8 show the results of the simulations in the three scenarios (based on age) at the doses of 100 mg/kg/day and 250 mg/kg/day in a population of normal subjects and in a population of subjects with mild, moderate, severe, and end stage renal disease. The left panels show the results of the simulations in the subjects with normal renal functions and the right panels show the results of the second stage of the analysis with the estimation of the dose correction factors.

The results of the simulations indicated that the total daily dose/kg in moderate, severe, and end stage renal disease patients should be reduced to reach comparable exposure to that expected in subjects with normal renal functions. Patients with mild renal impairment do not require dose adjustment as the expected exposure at the recommended doses do not differ from expected exposure in subjects with normal renal function. Furthermore, the results of the simulations suggested that no specific age related correction factor should be applied.

Based on the simulation results, the recommended initial and subsequent oral daily dosing regimens of carglumic acid are:
Mild renal impairment: no dose adjustment necessary;
Moderate renal impairment: daily dosage of 80 mg/kg/day to 200 mg/kg/day divided in two to four doses;
Severe renal impairment: daily dosage of 65 mg/kg/day to 160 mg/kg/day divided in two to four doses;
End stage renal disease: daily dosage of 60 mg/kg/day to 150 mg/kg/day divided in two to four doses.

### Example 11—Single Dose Study

Study Design

In this study, the pharmacokinetic of carglumic acid following a single oral dose of 80 mg/kg (or lower) in subjects with varying degree of impaired renal function was compared with matched, healthy controls with normal renal function. The safety and tolerability of a single oral dose of 80 mg/kg (or lower) of carglumic acid in subjects with normal and varying degree of impaired renal function was also evaluated in the study.

39 subjects using an adaptive design that included 3 groups of subjects with different degrees of renal impairment and two groups of control subjects with normal renal function were enrolled in the study based on the renal function at screening:
Group 1a: Healthy subjects with normal renal function (eGFR>90 mL/min/1.73 m²) (8 subjects)
Group 1b (As dose of Group 4 was less than 80 mg/kg): Healthy subjects with normal renal function (eGFR>90 mL/min/1.73 m²) (7 subjects)
Group 2: Mild renal impairment subjects (eGFR 60-89 mL/min/1.73 m²) (8 subjects)
Group 3: Moderate renal impairment subjects (eGFR 30-59 mL/min/1.73 m²) (8 subjects)
Group 4: Severe renal impairment subjects (eGFR<29 mL/min/1.73 m²) (8 subjects)

Based on safety and PK results from subjects with mild and moderate renal impairment (Groups 2 and 3), the dose could be adjusted to a lower dose. As a lower dose was required in Group 4, two groups of subjects with normal renal function (Group 1a and 1b) were enrolled. One group of normal renal function matched the pool of mild and moderate renal impaired subjects and received the same dose as these subjects and a second group of normal renal function matched the pool of severe renal impaired subjects and received the same dose as these subjects.

All subjects participated in one treatment period and received a single dose of carglumic acid in the fasting state. Healthy and mild renal impaired subjects were confined to the clinic from 10 hours prior to dosing until 120 hours after drug administration. The confinement period for moderate and severe renal impaired subjects was from 10 hours prior to dosing until 36 hours after drug administration. These subjects returned to the clinical site for morning PK blood sample collections. Outpatient urine collection was performed and pooled samples were brought to the clinic at the next return visits. The total duration of each subject's participation in the study was 7 days (Day −1 through the last PK sample taken on Day 6), not including the screening.

Subject Inclusion Criteria

Each subject enrolled was fully eligible according to the protocol criteria. All subjects: 1) Availability for the entire study period; 2) Motivated volunteer and absence of intellectual problems likely to limit the validity of consent to participate in the study or the compliance with protocol requirements; ability to cooperate adequately; ability to understand and observe the instructions of the physician or designee; 3) Male or female volunteer; 4) A female volunteer must meet one of the following criteria: if of childbearing potential—agrees to use one of the accepted contraceptive regimens from at least 28 days prior to the drug administration, during the study and for at least 60 days after the dose. An acceptable method of contraception includes one of the following: abstinence from heterosexual intercourse, systemic contraceptives (birth control pills, injectable/implant/insertable hormonal birth control products, transdermal patch); intrauterine device (with or without hormones), male condom with spermicide or male condom with a vaginal spermicide (gel, foam, or suppository). If of non-childbearing potential—should be surgically sterile (i.e. has undergone complete hysterectomy, bilateral oophorectomy, or tubal ligation) or in a menopausal state (at least one year without menses); 5) Volunteer aged of at least 18 years; 6) Volunteer with a body mass index (BMI)≥18.50 kg/m² and <42.00 kg/m²; 7) Light-, non- or ex-smokers. A light smoker is defined as someone smoking 10 cigarettes (or equivalent tobacco products) or less per day for at least 3 months before Day 1 of this study. An ex-smoker is defined as someone who completely stopped smoking for at least 6 months before Day 1 of this study; 8) Willingness to adhere to the protocol requirements as evidenced by the informed consent form (ICF) duly read, signed and dated by the volunteer.

Subjects with Normal Renal Function: 9) Clinical laboratory values within the laboratory's stated normal range; if not within this range, these must be without any clinical significance; 10) Have no clinically significant diseases captured in the medical history or evidence of clinically significant findings on physical examination and/or clinical laboratory evaluations (hematology, general biochemistry, electrocardiogram [ECG], and urinalysis); 11) If no dose adjustment is required for Group 4: Have to match by age (±10 years), weight (±15%) and gender (similar percentage ratio) to the pooled mean values of subjects with mild, moderate and severe renal impairment; Or if dose adjustment is required for Group 4: Two groups will be enrolled; in one group, have to match by age (±15 years), weight (±20%) and gender (similar percentage ratio) to the pooled mean values of subjects with mild and moderate renal impairment and in the other group, have to match by age (±15 years), weight (±20%) and gender (similar percentage ratio) to the pooled mean values of subjects with severe renal impairment; 12) Have an eGFR>90 mL/min/1.73 m² calculated using MDRD equation at screening.

Renally Impaired Subjects: 13) Considered clinically stable in the opinion of the Investigator; 14) Presence of mild renal impairment (eGFR 60-89 mL/min/1.73 m²), moderate renal impairment (eGFR 30-59 mL/min/1.73 m²), or severe renal impairment (eGFR≤29 mL/min/1.73 m²) calculated using MDRD equation at screening.

Subject Exclusion Criteria

The following subjects were excluded from the study. All subjects: 1) Females who are pregnant or are lactating; 2) History of significant hypersensitivity to carglumic acid or any related products (including excipients of the formulations) as well as severe hypersensitivity reactions (like angioedema) to any drugs; 3) Positive results to HIV Ag/Ab Combo, Hepatitis B surface Antigen (HBsAG (B) (hepatitis B)) or Hepatitis C Virus (HCV (C)) tests; 4) Significant history of drug dependency or alcohol abuse (>3 units of alcohol per day, intake of excessive alcohol, acute or chronic); 5) Females who are pregnant according to a positive pregnancy test; 6) Volunteers who took carglumic acid in the previous 28 days before Day 1 of this study; 7) Volunteers who took an Investigational Product (in another clinical trial) in the previous 28 days before Day 1 of this study; 8) Volunteers who have already participated in this clinical study; 9) Volunteers who donated 50 mL or more of blood in the previous 28 days before Day 1 of this study; 10) Donation of 500 mL or more of blood (Canadian Blood Services, Hema-Quebec, clinical studies, etc.) in the previous 56 days before Day 1 of this study.

Subjects with Normal Renal Function: 11) Seated pulse rate less than or equal 40 Beats per Minute (bpm) or more than 100 bpm at screening; 12) Seated blood pressure below 90/60 mmHg or higher than 140/90 mmHg at screening; 13) Any history of tuberculosis and/or prophylaxis for tuberculosis; 14) Presence of significant gastrointestinal, liver, or kidney disease, or any other conditions known to interfere with the absorption, distribution, metabolism, or excretion of drugs or known to potentiate or predispose to undesired effects; 15) History of significant gastrointestinal, liver or kidney disease that may affect drug bioavailability, including but not limited to cholecystectomy; 16) Presence of significant cardiovascular, pulmonary, hematologic, neurological, psychiatric, endocrine, immunologic or dermatologic disease; 17. Presence of out-of-range cardiac interval (PR<110 msec, PR>220 msec, QRS<60 msec, QRS>119 msec and QTc>450 msec for males and >460 msec for females) on the screening ECG or other clinically significant ECG abnormalities; 18) Use of any enzyme-modifying drugs, including strong inhibitors of cytochrome P450 (CYP) enzymes (such as cimetidine, fluoxetine, quinidine, erythromycin, ciprofloxacin, fluconazole, ketoconazole, diltiazem and HIV antivirals) and strong inducers of CYP enzymes (such as barbiturates, carbamazepine, glucocorticoids, phenytoin, rifampin and St John's Wort), in the previous 28 days before Day 1 of this study; 19) Positive screening of alcohol and/or drugs of abuse; 20) Any clinically significant illness in the previous 28 days before Day 1 of this study.

Renal Impaired Subjects: 21) Seated pulse rate less than 50 bpm or more than 110 bpm at screening; 22) Seated blood pressure below 90/50 mmHg or higher than 180/110 mmHg at screening; 23) Currently undergoing any method of dialysis; 24) History of renal transplant; 25) Presence or history of any disorder (including Parkinson disease) that could interfere with completion of the study based on the opinion of the Investigator; 26) History or presence, in the opinion of the Investigator, of significant clinically unstable respiratory, cardiovascular, pulmonary, hepatic, hematologic, gastrointestinal, endocrine, immunologic, dermatologic, neurologic, or psychiatric disease; 27) Have poorly controlled Type 1 or Type 2 diabetes as defined by Hemoglobin A1c>10%; 28) Require immunosuppressive medications for treatment of immune-mediated renal disease or kidney transplant recipients; 29) Evidence of renal carcinoma present at the time of screening; 30) Have relevant clinical laboratory abnormalities, including any elevation of alanine aminotransferase (ALT), aspartate aminotransferase (AST), or bilirubin at screening. If the investigator concludes that there is no safety risk for subjects with isolated laboratory abnormalities (e.g., those that do not reflect end-organ dysfunction; for example, elevated bilirubin in Gilbert's subjects) to participate in the study, such cases need to be discussed and approved by the sponsor's medical monitor prior to study enrollment; 31) Presence of clinically significant physical, laboratory, or electrocardiogram (ECG) finding that, in the opinion of the Investigator and/or sponsor, may interfere with any aspect of study conduct or interpretation of results; 32) Subjects with acute, unstable, or untreated significant medical conditions. Subjects requiring treatment for renal impairment or other chronic disease (e.g., well-controlled diabetes, hypertension) must be on a stable treatment plan (medicines, doses, and regimens) for at least 2 weeks (except insulin) prior to Day 1 and during the entire study. Small adjustments in the dosages of some concomitant medications may be permitted during the study, and will be discussed on a case-by-case basis. In all cases, the subjects' treatment history must be reviewed and their enrollment must be agreed to by both the investigator and the sponsor's medical monitor; 33) Positive screening of alcohol and/or drugs of abuse unless results can be explained by a prescription medication; 34) Concurrent use of medications known to affect the elimination of serum creatinine (e.g., BACTRIM® (trimethoprim/sulfamethoxazole) or TAGAMET® (cimetidine) and competitors of renal tubular secretion (e.g., probenecid) within 30 days prior to the first dose of study drug or anticipated need for these therapies through the last PK sample.

Formulation of CARBAGLU® (Carglumic Acid)

The active ingredient of the CARBAGLU® tablet is carglumic acid, and the excipients of the tablet include microcrystalline cellulose, sodium laurylsulfate, hypromellose, croscarmellose sodium, silica colloidal anhydrous, and sodium stearyl fumarate.

Schedule of Drug Administration

The CARBAGLU® (carglumic acid) 200 mg dispersible tables were administered orally as a single 80 mg/kg dose (or lower dose for severe renal impaired subjects and matched subjects with normal renal function) rounded to the nearest half tablet (i.e., 100 mg).

The tablets were dispersed in approximately 150 mL of non-carbonated water at room temperature and stirred with a spoon. The tablets do not dissolve completely in water and un-dissolved particles of the tablet may remain in the mixing container. The subject was asked to drink the prepared suspension. Thereafter, the glass was rinsed twice with approximately 50 mL of non-carbonated water at room temperature and administered to the subject, for a total of 250 mL of water administered. Dosing time was set to the start time of drinking the mixture. Dosing was completed within 5 minutes of the dosing start time.

Subjects were asked to fast overnight (no food or drink, except water) prior to dosing, for a minimum of 10 hours. Fasting continued for at least 4 hours following drug administration, after which a standardized lunch was served. A supper, a light snack and other meals was also served at appropriate times thereafter, but not before 9 hours after dosing.

Fluid intake other than water was controlled for housing period and for all subjects. Water were provided ad libitum until 1 hour pre-dose and were allowed ad libitum beginning 1 hour after the administration of the drug.

Prior and Concomitant Medication

Renal-impaired subjects were permitted to continue taking any prescription or over-the-counter (OTC) medication necessary for the management of their renal disease or other concurrent illness. The dosing schedules must have been stable for 2 weeks (except insulin) before Day 1 of the study and maintained throughout the study. Minor dose changes consistent with treatment practices were permitted at the discretion of the Investigator. All medications taken during the 14 days prior to dosing were recorded in the subject's Case Report Forms (CRF) and reviewed by the Investigator. When concomitant medications were administered, the indication, name, dose, route, and frequency were recorded in each subject's CRF.

For healthy subjects, in addition to the drugs prohibited as per the exclusion criteria, subjects were prohibited from taking any other prescription medications used with the intention to treat a condition for 28 days prior to the first dosing and during the study, unless judged differently by the qualified investigator or designee. Systemic contraceptives and hormone replacement therapy were permitted. Subjects were also prohibited from taking any over-the-counter (OTC) products for 7 days prior to the first dosing and during the study. They were specifically reminded that this includes cold preparations (containing ASA), acetylsalicylic acid (ASA), vitamins and natural products used for therapeutic benefits and antacid preparations. Vitamins used as nutritional supplements in non-therapeutic doses could be accepted, but had to be stopped at least 48 hours prior to the first dosing and during the study.

Study Procedures

Assessments for each study parts are summarized in Table 1 and indicated with an “X” when the assessments have to be performed. A complete physical examination was performed by a medically qualified and licensed individual as scheduled in Table 1. The physical examination included a review of the following: head and neck, heart, lungs, abdomen and general appearance.

TABLE 1
Schedule of Assessment for all subjects

| | | | | | | | | | End of | |
|---|---|---|---|---|---|---|---|---|---|---|
| | | | | | | | | | Study | |
| | Screening | Days | | | | | | | Tests | Follow- |
| | Day −28 | −1 | | | | | | | or ET^{a} | up call |
| Examination | to −1 | (Admission) | 1 | 2 | 3 | 4 | 5 | 6 | 6 | 9 (±3) |
| Review Inc/Exclusion Criteria & Medical | X | | | | | | | | | |
| History | | | | | | | | | | |
| Informed Consent | X | | | | | | | | | |
| Admission | | X | | | | | | | | |
| Study Drug Administration | | | X | | | | | | | |
| Clinic confinement for healthy and mild | | X | X | X | X | X | X | X | | |
| renal impaired subjects^{b} | | | | | | | | | | |
| Clinic confinement moderate and severe | | X | X | X | | | | | | |
| renal impaired subjects^{c} | | | | | | | | | | |
| Return visits for moderate and severe | | | | | X | X | X | X | | |
| renal impaired subjects^{c} | | | | | | | | | | |
| Demographics | X | | | | | | | | | |
| Concomitant Medication | X | X | X | X | X | X | X | X | X | X |
| Physical Examination | X | | | | | | | | X | |
| Vital Signs^{d} | X | | X | X | X | X | | | X | |
| Height, Weight, and BMI | X | X^{e} | | | | | | | | |
| 12-lead ECG for healthy and mild | X | | X | X | | | | | | |
| impaired subjects^{f} | | | | | | | | | | |
| 12-lead ECG for moderate and severe | X | | X | X | X | X | | | X | |
| impaired subjects^{g} | | | | | | | | | | |
| HIV Ag/Ab Combo, ABsAG (B) | X | | | | | | | | | |
| (Hepatitis B) and HCV (C) Tests | | | | | | | | | | |
| Drug and Alcohol Screen | X | X | | | | | | | | |
| Pregnancy test (females)^{h} | X | X | | | | | | | X | |
| Clinical Laboratory Evaluationsⁱ | X | X | | | | | | | X | |
| PK Blood Samples^{j} | | | X | X | X | X | X | X | | |
| Urine PK Collection^{j} | | | X | X | X | X | X | X | | |
| Outpatient urine collection instructions | | | | X | | | | | | |
| and diary distribution for moderate and | | | | | | | | | | |
| severe renal impaired subjects | | | | | | | | | | |
| Outpatient urine samples brought to the | | | | | X | X | X | X | | |
| clinic and diary verification for moderate | | | | | | | | | | |
| and severe renal impaired subjects | | | | | | | | | | |
| Adverse Events Recording^{k} | X | X | X | X | X | X | X | X | X | X |

^{a}Early Termination.
^{b}Healthy and mild renal impaired subjects will be confined to the clinic from 10 hours prior to dosing until 120 hours after drug administration.
^{c}Moderate and severe renal impaired subjects will be confined from 10 hours prior to dosing until 36 hours after drug administration. These subjects will return to the clinical site for morning PK blood sample collections (48, 72, 96 and 120 hours).
^{d}Vital signs (body temperature, pulse rate, and blood pressure) will be measured prior to dosing and at 1, 2, 3, 4, 5, 6, 8, 24, 48 and 72 hours after study drug administration.
^{e}Weight only.
^{f}A single 12-lead ECG will be performed prior to dosing and approximately 3 and 24 hours after study drug administration.
^{g}12-lead ECG will be performed prior to dosingand approximately 2, 3, 4, 6, 8, 24, 48, and 72 hours after study drug administration. The 12-lead ECG taken at 2, 3, 4, 6, 8 and 24 hours will be taken as triplicate.
^{h}At screening, a serum pregnancy test will be performed. At all other times, a urine pregnancy test will be done.
ⁱClinical laboratory tests (hematology, biochemistry, and urinalysis) will be performed in the evening prior to drug administration.
^{j}PK blood and urine samples will be collected according to schedule of PK assessments in the Pharmacokinetic Sampling section shown below.
^{k}AE reported from the ICF signed off until the follow-up phone call.

Safety assessments included physical examination, vital signs (blood pressure, heart rate, and body temperature), 12-lead ECG, laboratory tests (hematology, biochemistry, and urinalysis), and continuous AE monitoring. Body weight and height were measured at Screening. Body weight was also measured at admission and was used to calculate the dose to be administered.

Laboratory evaluations were performed as scheduled in Table 1. The physician in charge or designee assessed each abnormal value to determine if it is clinically significant. Clinical laboratory evaluations include general biochemistry: Sodium, potassium, chloride, glucose, blood urea nitrogen (BUN), creatinine, eGFR2, CLCr3, total bilirubin, alkaline phosphatase, AST, ALT and albumin; hematology: White cell count with differential (absolute values of neutrophil, lymphocyte, monocyte, eosinophil, and basophil), red cell count, hemoglobin, hematocrit, mean corpuscular volume (MCV), and platelets count; urinalysis: Color, appearance, specific gravity, pH, leukocyte, protein, glucose, ketones, bilirubin, blood, nitrite, urobilinogen (microscopic examination will only be performed if the dipstick test is outside of the reference range for leukocyte, blood, nitrite or protein); serology: HIV Ag/Ab Combo, HBsAg and hepatitis C (HCV); and urine drug screen: alcohol, amphetamines, barbiturates, cannabinoids (THC), cocaine, opiates and (PCP) phencyclidine. A pregnancy test was performed on female subjects as specified in Table 1.

Twelve-lead electrocardiogram (ECG) was performed as specified in Table 1. For moderate and severe impaired subjects, the 12-lead ECG taken at 2, 3, 4, 6, 8 and 24 hours was taken as triplicate. Three separate 10-second ECG recordings were collected at intervals of approximately 1 minute apart. The mean of the 3 readings was used as the final measure of the ECG parameters. On days ECGs are conducted, they were scheduled prior to blood draws that occurred for that day, as the blood draws could impact the ECG reading.

Pharmacokinetic Sampling

Blood samples were collected into Na heparin Vacutainers by direct venipuncture. However, as an option to the volunteer or if judged necessary by the clinical staff, blood samples were collected from an indwelling cannula (stylet catheter that requires no flushing), which were placed in the forearm vein of the subject. Blood samples were collected in one tube of 6 mL each.

The complete schedule is presented here. The pharmacokinetic blood samples were collected from the subjects prior to dosing, and 0.5, 1, 1.5, 2, 3, 4, 6, 8, 12, 16, 24, 36, 48, 72, 96 and 120 hours post-dose. The pharmacokinetic urine samples were collected from the subjects prior to dosing, and 0-6, 6-12, 12-24, 24-36, 36-48, 48-72, 72-96 and 96-120 hours post-dose. The time of blood sample collection was calculated according to the drug administration schedule.

The total volume of blood withdrawn, including ˜22 mL required for screening, pre-dose and end of study tests, did not exceed 124 mL per subject.

During the study, a baseline urine sample was collected before drug administration. After dosing, urine samples were collected over 120 hours over 8 collection intervals. The total volume of urine collected at each interval was measured (weight) and two containers (˜5 mL each) were preserved.

Bioanalytical Methods

Plasma and urine concentrations were measured according to validated bioanalytical methods. Samples from all subjects who received the investigational product were assayed. The validated range was truncated when less than 5 calibrants appeared to be below the expected Cmax, provided that this new calibration curve range was validated prior to use.

Where possible, all samples from each subject were analyzed on the same standard curve. Quality control samples were distributed through each batch of study samples assayed. Samples with drug concentrations greater than the upper limit of quantification (ULQ) of the assay range were diluted with the appropriate drug-free biological fluid and re-assayed; those which were below the lower limit of this range were reported as being below limit of quantitation concentrations (BLQ).

The PK parameters are presented in Table 2. Below limit of quantitation concentrations (coded BLQ) were treated as zero for all PK analyses. The evaluation of plasma PK parameters took into account all actual PK sampling times.

The PK parameters were estimated using a non-compartmental approach with a log-linear terminal phase assumption. The trapezoidal rule was used to estimate the area under the curve, and the terminal phase was estimated by maximizing the coefficient of determination estimated from the log-linear regression model. Disposition parameters (AUC0-∞, AUC0-T/∞, λZ, Thalf, CLTOT/F and VD/F) were not estimated for individual concentration-time profiles where the terminal log-linear phase could not be reliably characterized. PK analyses were generated using PHOENIX® WINNONLIN® Version 8.0 (or higher).

TABLE 2
Pharmacokinetic Parameters

| PK | |
|---|---|
| Parameter | Definition |
| Cₘₐₓ | Maximum observe plasma concentration |
| Tₘₐₓ | Time of maximum observed plasma concentration; if it |
| | occurs at more than one time point, Tₘₐₓ is defined as the |
| | first time point with this value |
| AUC_{0-T} | Cumulative area under the plasma concentration time curve |
| | calculated from 0 to T_{LQC} using the linear trapezoidal |
| | method, where T_{LQC} represents time of last observed |
| | quantifiable plasma concentration |
| AUC_{0-∞} | Area under the plasma concentration time curve |
| | extrapolated to infinity, calculated as AUC_{T} + C_{LQC}/λ_{z}, |
| | where C_{LQC} is the measured concentration at time T_{LQC} |
| AUC_{0-T/∞} | Relative percentage of AUC_{0-T} with respect to AUC_{0-∞} |
| λ_{z} | Apparent elimination rate constant, estimated by linear |
| | regression of the terminal linear portion of the log |
| | concentration versus time curve |
| T_{half} | Terminal elimination half-life, calculated as 1n(2)/λ_{z} |
| CL_{TOT}/F | Apparent Total Plasma Clearance, calculated as |
| | carglumic acid dose/AUC_{0-∞} |
| V_{D}/F | Apparent Volume of Distribution, calculated as |
| | carglumic acid dose/λ_{z}*AUC_{0-∞} |
| Ae(0-t) | Amount excreted in urine (Total carglumic acid |
| | concentration * Volume of Urine) |
| fe | Fraction of dose excreted in urine (Ae/carglumic |
| | acid dose) |
| CLᵣ | Renal Clearance (Ae(0-t/AUC_{0-T}) |

Descriptive statistics were calculated for plasma concentrations at each individual time point and for all PK parameters. The individual plasma concentration/time profiles were presented using the actual sampling times whereas the mean plasma concentration/time profiles was presented using the theoretical sampling times. Only subjects included in the PK analysis sets were included in the PK analysis.

Regression Analysis

The eGFR and Cockcroft-Gault estimate of the creatinine clearance at baseline was used as separate measures of renal function for a regression analysis to evaluate the relationships between estimated renal function and the PK parameters. For each PK parameter, a regression analysis was performed to assess the impact of impaired eGFR and creatinine clearance (CLCR), using a regression model of the form α+β*eGFR (or CLCR)+ε where the errors (ε) will be assumed to be independent and normally distributed with mean zero and variance σ². The parameter β represents the correlation between the relevant PK parameter and eGFR (or CLCR) which was treated as a continuous variable. The hypothesis of the slope of trend being different from zero was assumed if the two-sided test of the nullity of the parameter β was statistically significant at the 5% level.

Analysis of Variance

Based on the regression analysis described in the previous section, if the slope of trend of the regression analysis between a PK parameter and the estimated renal function (eGFR or CLCR) was found to be significant (p<0.05), an analysis of variance (ANOVA) was performed to assess the difference in the PK parameter among the renal function groups.

The renal function (normal, mild, moderate and severe) was entered as a fixed effect in the ANOVA model. Pairwise comparisons of renal function groups were generated using the Tukey-Kramer's procedure of adjustment for multiple comparisons (if more than 2 renal function groups) and statistical significance was assessed at the two-sided 5% level. The ratio of geometric LS means (of each renal function group being compared), with a corresponding 90% confidence interval (adjusted using Tukey-Kramer's procedure if applicable), was computed. Heterogeneity of variance among groups was assumed.

First, subjects with mild and moderate renal impairment were enrolled. Samples were assayed and PK was performed. Thereafter, Group 4 (Severe Renal Impairment) was enrolled. Based on safety and PK results from subjects with mild and moderate renal impairment, the dose could be adjusted to a lower dose, if required. If a lower dose was required in Group 4, two groups of subjects with normal renal function (Group 1a and 1b) could be enrolled. One group of normal renal function would match the pool of mild and moderate renal impaired subjects and received the same dose as these subjects and a second group of normal renal function would match the pool of severe renal impaired subjects and received the same dose as these subjects. If the dose does not need to be adjusted for Group 4 then, only one group of 8 subjects with normal renal function was enrolled and matched the pool of mild, moderate and severe renal impaired subjects.

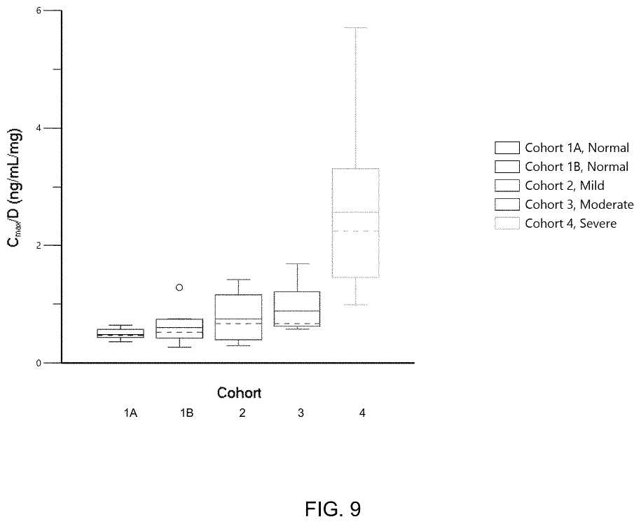

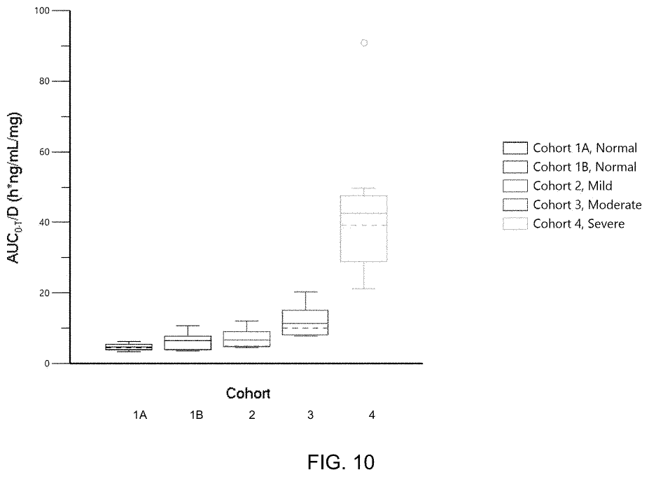

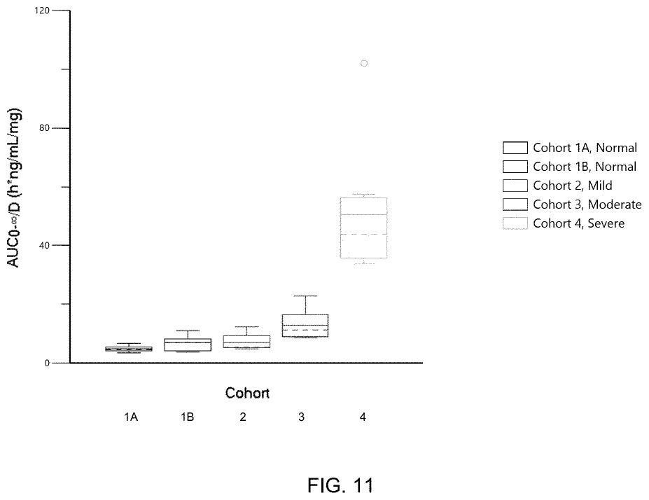

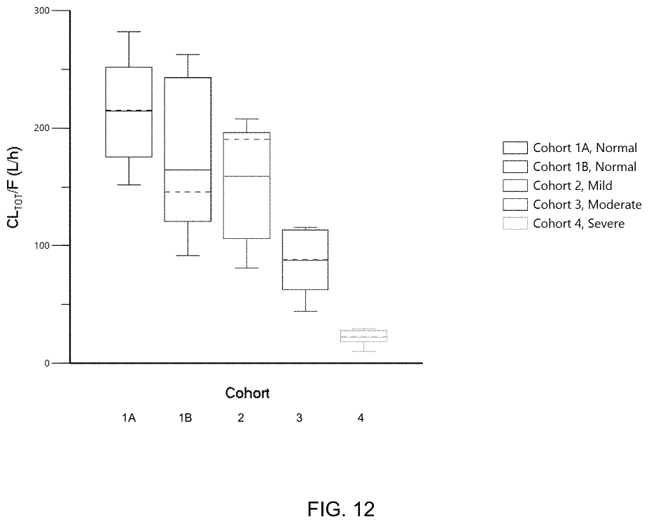

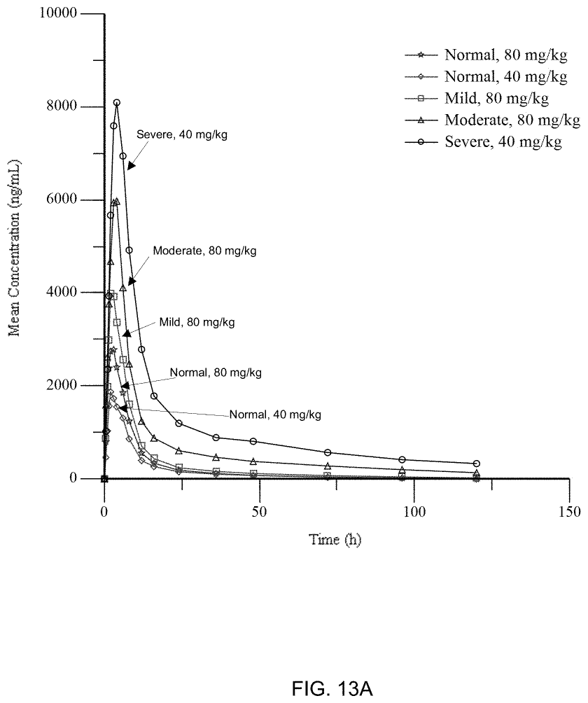

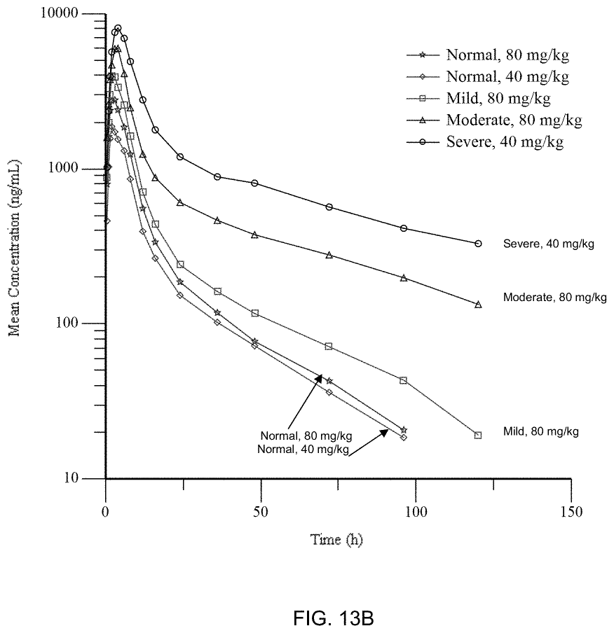

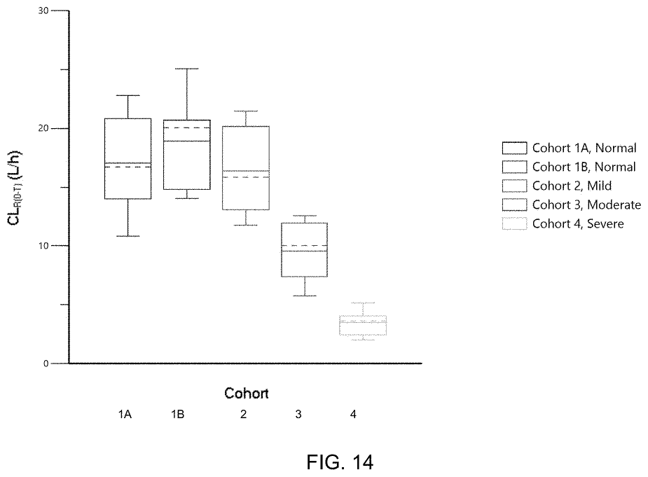

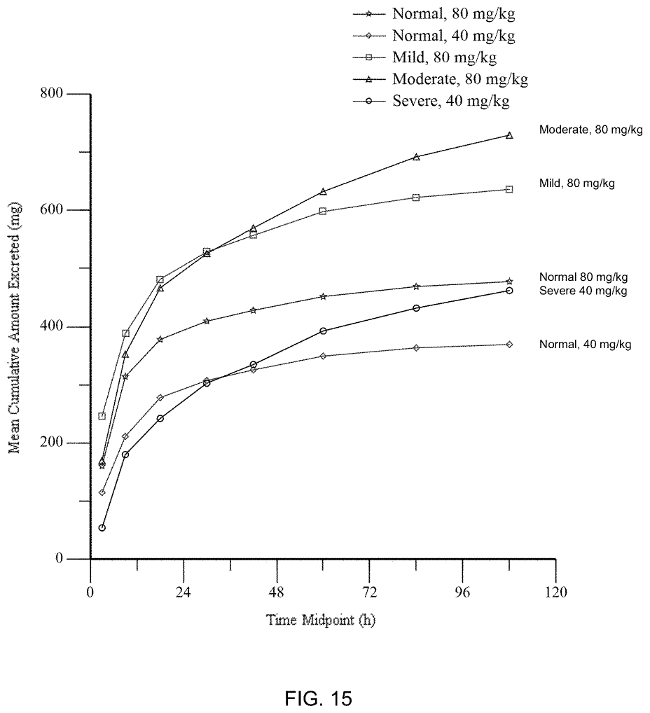

The plasma PK results are demonstrated in FIGS. 9-13. The urine PK results are demonstrated in FIGS. 14-15. Based on these PK results, the adjusted doses due to renal insufficiency were calculated to be:

For the acute treatment of hyperammonemia:
Mild renal impairment: no dose adjustment necessary
Moderate renal impairment: daily dosage of 50 mg/kg/day to 125 mg/kg/day divided in two to four doses
Severe renal impairment: daily dosage of 15 mg/kg/day to 60 mg/kg/day divided in two to four doses
End stage renal disease: daily dosage of 15 mg/kg/day to 60 mg/kg/day divided in two to four doses

For the chronic treatment of hyperammonemia:
Mild renal impairment: no dose adjustment necessary
Moderate renal impairment: daily dosage of 5 mg/kg/day to 50 mg/kg/day divided in two to four doses
Severe renal impairment: daily dosage of 2 mg/kg/day to 25 mg/kg/day divided in two to four doses
End stage renal disease: daily dosage of 2 mg/kg/day to 25 mg/kg/day divided in two to four doses

### Example 12—Phase 1 Safety and PK Study in Healthy Subjects of a New IV Formulation of Carbaglu Administered at Single Ascending Doses and at Different Infusion Durations and Determination of the Absolute Bioavailability of the Already Marketed Reference Oral Formulation (CARIV-A-001)

This study was conducted in healthy male or female volunteers, at a single site to assess the safety and PK profile of an IV formulation of Carbaglu and to determine the absolute bioavailability of the current marketed oral formulation of Carbaglu.

The study included three parts: Part A aimed at determining the maximum tolerated loading dose and characterizing the PK profile of CGA when administered by the IV route over a 20-minute infusion period, Part B aimed at comparing the bioavailability of CGA between the oral and the IV routes of administration, and Part C aimed at determining the safety of the IV route of administration and determining exposure over infusion periods of up to 24 hours in order to establish potential steady-state therapeutic levels.

The levels of CGA in plasma and urine were analysed using a fully validated method, which included LC-MS/MS analysis. The PK data were analysed based on a non-compartmental model. A model-based approach was implemented for the characterization of the PK properties of the IV formulation in order to estimate the level of PK exposure after different doses administered with different duration of infusion. The model outcomes provided rationales for supporting decision-making through the dose escalation process. The PK parameters were determined using SAS (version 9.3), and modelling of the PK data was performed using NONMEM (version 7.3, ICON Solutions).

### PART A: Mono-Centre, Randomized, Successive Groups, Double-Blind, Placebo-Controlled Study in Healthy Male and Female Subjects

Part A was a randomized, double-blind, placebo-controlled study conducted in three successive groups of 8 individuals, for a total of 24, including 12 male and 12 female subjects, of whom 23 were Caucasians and one was Black. Each cohort included a minimum of 3 and maximum of 5 subjects of each of the two genders. Age ranged from 20 to 44 years (mean±SD=29.2±6.0), height from 154 to 185 cm (mean±SD=171.7±9.5), body weight from 52.4 to 87.6 kg (mean±SD=68.0±9.1) and body mass index (BMI) from 19.9 to 28.8 kg/m² (mean±SD=23.0±2.1).

Three loading doses of CGA over 20 minutes were tested by IV administration, 3.5 mg/kg, 5.0 mg/kg and 7.5 mg/kg. For each of these three doses, 6 subjects were administered CGA and 2 received the placebo.

Plasma concentrations of CGA were measured at 18 timepoints, including a sample preceding the start of the administration, and then between 10 minutes and 48 hours following the initiation of the IV infusion.

The PK analysis of Carbaglu was conducted by using standard non-compartmental approach.

A biexponential decay was observed and the terminal half-life value was properly determined in all subjects at all doses. The extrapolated AUC was always found to be less than 15% (on average below 7.8% in each cohort) indicating that the data collected were appropriate for characterizing the Carbaglu PK. Inter-individual variability of PK parameters, as expressed by coefficient of variation (CV) %, was moderate (between 5.6 and 24.4% for Cₘₐₓ and 10.1 and 16.6% for AUC). The comparison of the half-live values across dose groups did not show any dose-related changes: the mean half-life value was 22 h (between 20.6 and 24.8 h in each cohort). Total body clearance remains constant through the tested dose range: mean value 0.32 L·h/kg in each cohort. A large volume of distribution was estimated as volume of distribution (V_{d}) (mean between 9.3 and 11.2 L/kg in each cohort) due to the long half-life observed; while a lower Vₛₛ was calculated (mean between 2.21 and 2.39 L/kg).

The comparison of the Carbaglu exposure at three dose levels indicated a proportional increase of plasma levels with the increase of the dose. The AUC_{inf}, AUCₗₐₛₜ, and Cₘₐₓ were shown to increase linearly for doses in the range of 3.5 mg/kg to 7.5 mg/kg using either linear of power modelling approach. AUC_{inf}, AUCₗₐₛₜ, and Cₘₐₓ mean values (CV %), at 3.5 mg/kg, 5.0 mg/kg, and 7.5 mg/kg, are summarized in Table 10.

TABLE 10
Descriptive Statistics of the PK Parameters by Dose

| | Dose | | |
|---|---|---|---|
| PK Parameter | 3.5 mg/kg | 5.0 mg/kg | 7.5 mg/kg |
| Cₘₐₓ | Mean = 11417 | Mean = 15003 | Mean = 22917 |
| (ng/mL) | S.E.M. = 260 | S.E.M. = 1495 | S.E.M. = 1316 |
| | Median = 11350 | Median = 15550 | Median = 23250 |
| | Range = 10700-12300 | Range = 8420-19100 | Range = 18200-26500 |
| AUCₗₐₛₜ₋ₜᵢₘₑₚₒᵢₙₜ | Mean = 10159 | Mean = 15268 | Mean = 22638 |
| (μg · h/L) | S.E.M. = 401 | S.E.M. = 1098 | S.E.M. = 1283 |
| | Median = 10,113 | Median = 15622 | Median = 22069 |
| | Range = 8988-11488 | Range = 11459-19307 | Range = 19566-27818 |
| AUC_{24 h} | Mean = 9846 | Mean = 14755 | Mean = 21650 |
| (μg · h/L) | S.E.M. = 312 | S.E.M. = 1131 | S.E.M. = 1216 |
| | Median = 9687 | Median = 14917 | Median = 20744 |
| | Range = 8988-11007 | Range = 11056-19307 | Range = 19209-26910 |
| AUC_{inf} | Mean = 11030 | Mean = 16132 | Mean = 23897 |
| (μg · h/L) | S.E.M. = 454 | S.E.M. = 1091 | S.E.M. = 1296 |
| | Median = 10943 | Median = 16646 | Median = 23226 |
| | Range = 9742-12540 | Range = 12284-19951 | Range = 20168-29127 |
| T_{1/2} | Mean = 20.6 | Mean = 21.6 | Mean = 24.8 |
| (h) | S.E.M. = 4.6 | S.E.M. = 2.3 | S.E.M. = 3.4 |
| | Median = 21.1 | Median = 21.4 | Median = 25.5 |
| | Range = 6.6-36.1 | Range = 12.8-29.3 | Range = 15.3-35.4 |
| Clearance | Mean = 0.32 | Mean = 0.32 | Mean = 0.32 |
| (Cl) | S.E.M. = 0.01 | S.E.M. = 0.02 | S.E.M. = 0.02 |
| (L/h/kg) | Median = 0.32 | Median = 0.30 | Median = 0.32 |
| | Range = 0.28-0.36 | Range = 0.25-0.41 | Range = 0.26-0.37 |
| Renal | Mean = 0.26 | Mean = 0.23 | Mean = 0.26 |
| Clearance | S.E.M. = 0.02 | S.E.M. = 0.02 | S.E.M. = 0.02 |
| (RCl) | Median = 0.25 | Median = 0.22 | Median = 0.25 |
| (L/h/kg) | Range = 0.20-0.30 | Range = 0.19-0.31 | Range = 0.21-0.32 |
| Volume of | Mean = 9.28 | Mean = 9.94 | Mean = 11.23 |
| Distribution | S.E.M. = 1.92 | S.E.M. = 1.21 | S.E.M. = 1.55 |
| (V_{d}) (L/kg) | Median = 8.75 | Median = 10.21 | Median = 10.79 |
| | Range = 3.26-15.47 | Range = 4.62-12.75 | Range = 7.02-17.54 |

The analysis of the urinary excretion data indicated that the percent of dose excreted in urine remain stable at ˜75% across the doses evaluated. The renal clearance remains also stable across doses with a value of ˜0.25 L*h/kg and with an inter-individual variability (CV %) not exceeding 22%.

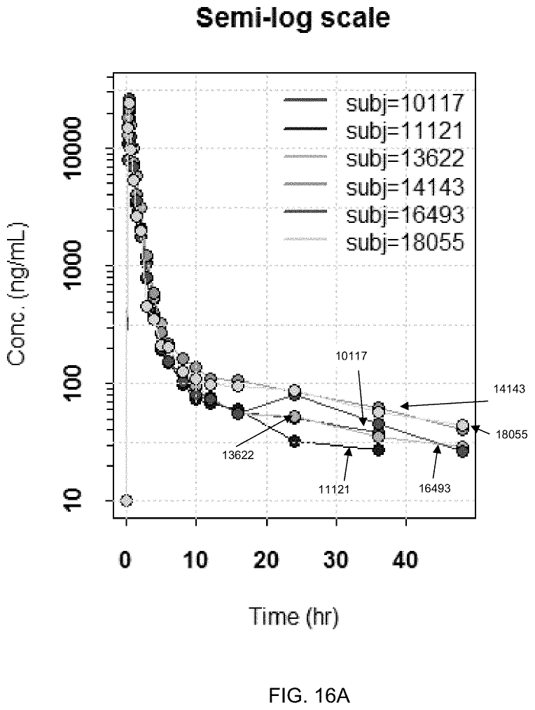

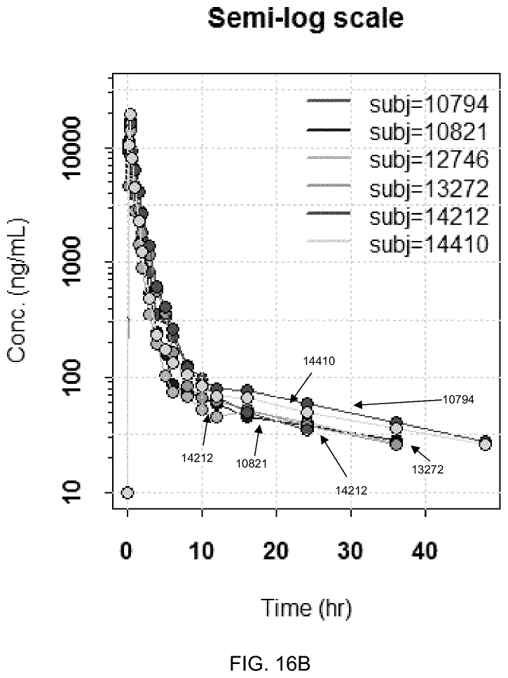

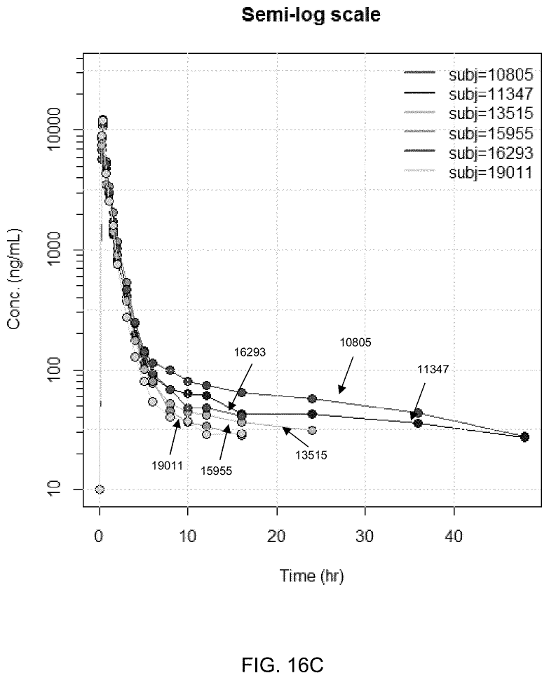

Overall analysis on the three groups showed that the median volume of distribution determined after IV administration is 10.2 L/kg. The median total clearance is 0.354 L/min (0.241 to 0.502 L/min) and 75% (59 to 86%) of the dose is excreted in the urine over 24 hours as parent compound. The median renal clearance is 265 mL/min (183 to 467 mL/min) inter-individual variability (CV %) is 17% (FIG. 16).

In conclusion, levels of up to 26,500 ng/mL were observed in healthy volunteers following IV administration over 20 minutes, showing linearity of kinetics after IV administration and that the main excretion route of parent compound is through renal clearance.

The results from Part A of the IV study were included into the dossier of the oral product, because they are relevant to define the PK of product, in particular the findings that the product is prevalently cleared by kidney and that the clearance remain constant through all the range of doses showing linear kinetics, even at higher concentration than those reached by oral route. Moreover, the Volume of distribution was properly determined.

### Part B: Mono-Centre, Randomized, Two-Period, Two-Sequence, Cross-Over Study in Healthy Male and Female Subjects

Part B was a randomized, double-blind, cross-over, two-sequence study. It was conducted in 12 healthy male and female subjects.

In two treatment periods, separated by a washout of at least two weeks, subjects received a single administration of the study medication in fasting conditions, either as an IV loading dose infusion of Rec0/0491 over 2 hours (started with 6 mg/kg/h then reduced to 4.0 mg/kg/h), or an oral tablet administration of Carbaglu® (100 mg/kg).

Blood samples for PK measurements were collected at 19 timepoints for the IV administration and 17 timepoints for the oral administration. This included a sample prior to intake of the study medication and the period ranging from 20 minutes to 72 hours following the initiation of the IV infusion, and the period ranging from 30 minutes to 72 hours following oral intake. Urine was also collected for PK measurements, at the following intervals relative to the time of administration of the study medication: 0-6, 6-12, 12-24, 24-36 and 36-48 hours.

Twenty-five healthy subjects were randomized, 13 male and 12 female subjects. Thirteen subjects were withdrawn from the study of whom 3 due to AE (including a SAE), 1 due to Investigator's decision due to technical issue during administration and 9 subjects withdrew informed consent after the 1^{st} period completed as they couldn't wait for several months because of study suspension (study was suspended after 2 AE assessed as new event (“fait nouveau”) for further investigations). Twelve healthy subjects (7 females and 5 males) completed two study periods, 8 were Caucasians, 2 were Black, and 2 were Asian. Age ranged from 24 to 44 years (mean±SD=32.8±6.5), height from 157 to 184 cm (mean±SD=171.7±9.0), body weight from 58.2 to 84.1 kg (mean±SD=69.2±8.4) and body mass index (BMI) from 19.0 to 28.3 kg/m² (mean±SD=23.5±2.6).

The PK analysis of Carbaglu was conducted on all subjects showing quantifiable plasma levels using standard non-compartmental approach.

A biexponential decay was observed and the terminal half-life value was properly determined in all subjects at all doses. The mean extrapolated AUC was always found to be less than 10% indicating that the data collected were appropriate for characterizing the Carbaglu PK after oral and IV administration. Inter-individual variability of the PK parameters, as expressed by CV %, was similar for the IV and oral formulations. The comparison of the half-live values across formulations indicated that the half-life of the oral formulation was slightly lower than the half-life of the IV formulation. The descriptive statistics on untransformed and dose normalized Cₘₐₓ, AUCₗₐₛₜ, AUC_{inf}, Half-life (HL), and Clearance (CL) values presented in Table 11.

TABLE 11
Descriptive statistics on Cmax, AUClast, AUCinf, Half-life, and CL
Untransformed Parameter Values

| | Dose | | | | Std | Std | | | |
|---|---|---|---|---|---|---|---|---|---|
| Treatment | (mg/kg) | N | Variable | Mean | Error | Dev | Median | Min | Max |
| IV | 8 | 10 | AUCinf | 24500.69 | 1613.30 | 5101.71 | 23713.71 | 16949.96 | 35564.90 |
| | | | aucL | 22856.89 | 1649.30 | 5215.55 | 21961.07 | 15713.03 | 34579.31 |
| | | | Cmax | 8613.00 | 558.17 | 1765.08 | 8765.00 | 5400.00 | 11700.00 |
| | | | HL | 30.64 | 2.72 | 8.60 | 28.81 | 22.85 | 52.95 |
| | | | CL | 0.339 | 0.021 | 0.068 | 0.338 | 0.225 | 0.472 |
| | 12 | 2 | AUCinf | 53814.72 | 8813.31 | 12463.90 | 53814.72 | 45001.41 | 62628.03 |
| | | | aucL | 52149.22 | 8868.18 | 12541.51 | 52149.22 | 43281.03 | 61017.40 |
| | | | Cmax | 21450.00 | 2050.00 | 2899.14 | 21450.00 | 19400.00 | 23500.00 |
| | | | HL | 34.91 | 7.68 | 10.86 | 34.91 | 27.23 | 42.59 |
| | | | CL | 0.229 | 0.038 | 0.053 | 0.229 | 0.192 | 0.267 |
| OR | 100 | 12 | AUCinf | 31425.49 | 2149.57 | 7446.34 | 30211.28 | 21852.27 | 45696.37 |
| | | | aucL | 29807.38 | 2144.60 | 7429.11 | 29088.84 | 20030.59 | 44250.17 |
| | | | Cmax | 3284.17 | 321.07 | 1112.23 | 2940.00 | 1950.00 | 5620.00 |
| | | | HL | 24.60 | 1.57 | 5.42 | 23.71 | 17.56 | 35.31 |
| | | | CL/F | 3.335 | 0.209 | 0.723 | 3.311 | 2.188 | 4.576 |

Dose normalized Parameter Values

| | | Std | Std | | | |
|---|---|---|---|---|---|---|
| Variable | Mean | Error | Dev | Median | Min | Max |
| refAUCinf | 3299.58 | 247.85 | 858.59 | 3162.4 | 2118.74 | 5219 |
| testAUCinf | 314.25 | 21.5 | 74.46 | 302.11 | 218.52 | 456.96 |
| refAUClast | 3105.22 | 255.41 | 884.77 | 2840.05 | 1964.13 | 5084.78 |
| testAUClast | 298.07 | 21.45 | 74.29 | 290.89 | 200.31 | 442.5 |
| refCmax | 1195.1 | 100.71 | 348.86 | 1148.13 | 675 | 1958.33 |
| testCmax | 32.84 | 3.21 | 11.12 | 29.4 | 19.5 | 56.2 |
| refHalf_life | 31.351 | 2.48 | 8.6 | 28.81 | 22.85 | 52.95 |
| testHalf_life | 24.6 | 1.56 | 5.42 | 23.71 | 17.56 | 35.31 |
| refCL | 0.038 | 0.003 | 0.012 | 0.04 | 0.016 | 0.059 |
| testCL | 0.033 | 0.002 | 0.007 | 0.033 | 0.022 | 0.046 |

The mean absolute bioavailability, evaluated by comparing the dose normalized AUC_{inf} parameter values, of the oral vs IV formulation in the 12 subjects receiving the two formulation, was F=9.87% (±2.38). This value is consistent with the ratio between the % of dose excreted in the urine after oral and IV administration (Table 12).

TABLE 12
Estimated absolute bioavailability of the oral vs
IV formulation computed using the arithmetic and
the geometric mean of the individual bioavailability

| | Mean | SD | GeoMean | GeoSD |
|---|---|---|---|---|
| F % | 9.87 | 2.38 | 9.57 | 2.68 |

The 90% confidence limits of the geometric mean of the ratio between Test (oral data) and Reference (IV data) for Cₘₐₓ, AUCₗₐₛₜ, AUC_{inf} and half-life were computed for the 12 subjects receiving both oral and IV formulations. The 90% confidence limits were compared to the reference limits of 0.8 and 1.25 representing the equivalence criterion. The results of the analysis indicated that the administration of loading dose of 4 mg/kg/h or 6 mg/kg/h by IV infusion over 2 hours and an oral tablet of 100 mg/kg administration of Carbaglu® provided a non-equivalent level of exposure of Carbaglu®. Absolute bioavailability 90% confidence intervals (CI) were between 8.30 and 11.03%. Linearity of PK was shown in Part A of this trial up to 26.5 μg/mL.

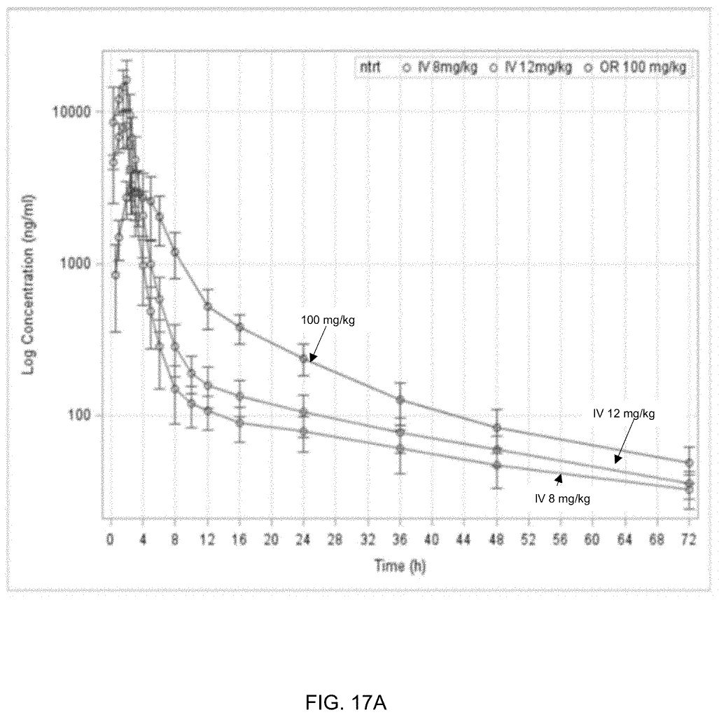

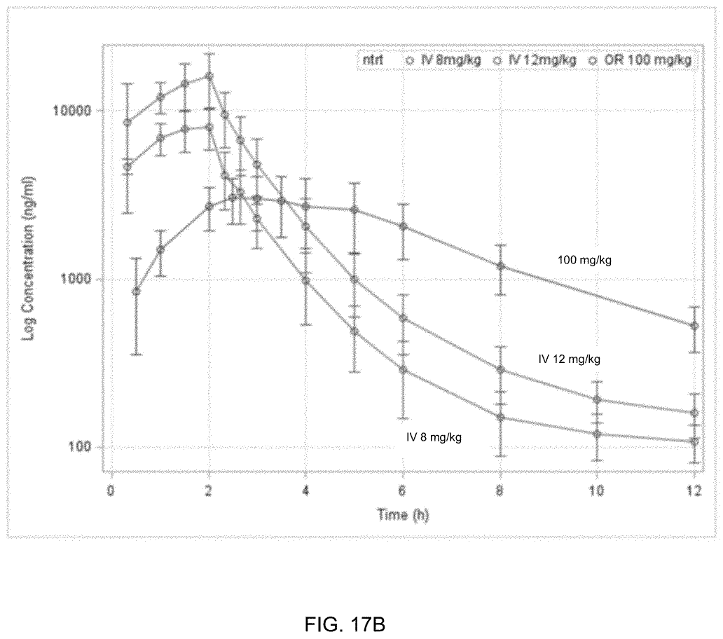

The mean Carbaglu plasma concentrations (±SD) vs. nominal time by treatment and dose in log-linear scale are reported in FIG. 17.

The analysis of the urinary excretion data indicated that the percent of dose excreted in urine after IV administration was 82% consistently with the results obtained in the Part A of the study, while after oral administration was 7.5%, which is consistent with the IV data taking into account an oral bioavailability of about 10%. The renal clearance value after IV administration of ˜0.30 L*h/kg was also consistent with the values found in the Part A of the study.

The total clearance after oral administration corrected by the bioavailability value of F=9.87% was 0.35 (L/hr/Kg). This value is consistent with total clearance estimated after IV administration of 0.31 (L/hr/Kg). The renal clearance after oral and IV administration are consistent each other, showing that it is not influenced by the route of administration.

### Part C: Mono-Centre, Randomized, Successive Groups, Double-Blind, Placebo-Controlled Study in Healthy Male and Female Subjects

Part C was a randomized, double-blind, placebo-controlled study. It was conducted in five successive cohorts of 8 healthy male or female subjects each, for a total of 40 subjects. For each of the five cohorts, 6 subjects were administered CGA and 2 subjects received placebo. The study medication was administered IV, first as a loading dose and then as a maintenance dose. The dosage regimen was determined based both on the results of Part A and PK modelling. This included determining the starting loading dose based on the results of Part A, and determining the maintenance dose in order not to exceed the highest AUC of 30.7 μg·h/mL previously observed in HVs following oral administration. The maximum dose to be administered was established in order not to exceed the exposure previously observed in a nonclinical safety study conducted in rats, the said exposure corresponding to a Cₘₐₓ value of 52 μg/mL and an AUC value of 258 μg·h/mL.

For the first three cohorts, three ascending doses of the study medication were administered IV as loading doses of 2.0, 3.0, and 3.6 mg/kg for 20 minutes, respectively, followed by maintenance doses of 2.5, 3.75 and 4.5 mg/kg/h given by IV infusion over a period of 0.3 hours and 40 minutes. For the fourth cohort, a dose of the IV formulation of CGA determined as being well tolerated was administered as a loading dose by IV infusion over 20 minutes, followed by a maintenance dose administered by IV infusion over a period of up to 12 hours. The 5^{th} cohort received the study medication at the same dose and under the same conditions as the 4^{th} cohort, except for the period during which the maintenance dose was administered, which lasted up to 24 hours.

Blood sample for PK measurements were collected at 18 timepoints for the first three cohorts, and 20 timepoints for the 4^{th} and 5^{th} cohorts. These included a sample preceding the administration of the study medication, and the period from 20 minutes to 48 hours following the initiation of the IV infusion. Urine was also collected for PK measurements at the following intervals relative to the time of initiation of the IV infusion: 0-6, 6-12, 12-24, 24-36, and 36-48 hours.

Forty healthy subjects (29 male and 11 female subjects) were randomized. All subjects completed the study. Thirty-two subjects were Caucasians, 4 were Black, and 4 were categorized in other ethnic origin. Age ranged from 20 to 44 years (mean±SD=30.0±6.9), height from 160 to 193 cm (mean±SD=173.1±8.0), body weight from 48.2 to 90.3 kg (mean±SD=70.9±10.0) and body mass index (BMI) from 18.8 to 28.6 kg/m² (mean±SD=23.7±2.9).

The PK analysis of Carbaglu was conducted on all measurements showing quantifiable levels using standard non-compartmental approach.

A biexponential decay was observed and the terminal half-life value was properly determined in all subjects at all doses. The mean extrapolated AUC in each group was always found to be less than 8% indicating that the data collected were appropriate for characterizing the Carbaglu PK. Inter-individual variability of PK parameters, as expressed by CV %, was consistent with the values found in the part A of the study.

The comparison of the Clearance, Volume distribution (Vd) and Half-Life values across treatment groups indicated that Carbaglu PK remains stables irrespectively of the dose and dosage regimen used. The average total body clearance, Vd, and Half-Life in the five treatment groups were: 0.320 L/h/Kg, 13.042 L/Kg, and 28.448 h, respectively. (Table 13).

TABLE 13
Descriptive statistics on Clearance, Vd and
Half-Life in the five treatment groups

| | | | Std | Std | | | |
|---|---|---|---|---|---|---|---|
| Variable | N | Mean | Error | Dev | Median | Min | Max |
| Cl (L/hr/Kg) | 27 | 0.320 | 0.013 | 0.065 | 0.316 | 0.192 | 0.438 |
| Vd (L/Kg) | 27 | 13.042 | 0.672 | 3.493 | 12.802 | 3.897 | 19.689 |
| Half-life (hr) | 27 | 28.448 | 1.247 | 6.478 | 28.533 | 13.598 | 39.987 |

The comparison of the renal clearance and the % of the dose excreted values across treatment groups indicated that Carbaglu urinary excretion remains stable irrespectively of the dose and dosage regimen used. The average renal clearance and the % of the dose excreted in the five treatment groups were: 0.290 L*h/kg and 84.478%, respectively (Table 14).

TABLE 14
Descriptive statistics on renal clearance and %
of the dose excreted in the five treatment groups

| | | | Std. | Std. | | | |
|---|---|---|---|---|---|---|---|
| Variable | N | Mean | Error | Dev | Median | Min | Max |
| Renal | 27 | 0.290 | 0.016 | 0.082 | 0.299 | 0.116 | 0.510 |
| Clearance | | | | | | | |
| (L/hr/Kg) | | | | | | | |
| Ae (%) | 27 | 84.478 | 3.049 | 15.842 | 86.924 | 38.756 | 111.412 |

The comparison of the Carbaglu exposure at three dose levels in group 1, 2, and 3 indicated a proportional increase of plasma levels with the increase of the dose. The AUC_{inf}, AUCₗₐₛₜ, and Cₘₐₓ increased linearly with the total doses (11.15 mg/kg to 20.07 mg/kg) infused over 4 hours using either linear of power modelling approach.

The results of the steady-state assessment indicated that the Carbaglu exposure initially reached (at 2 hours for group 1, 2, and 3 and at 4 hours for group 4 and 5) can be maintained for 4 hours for group 1 to 3, for 12 hours for group 4, and for 24 hours for group 5 with the doses and the infusion durations evaluated in this trial.

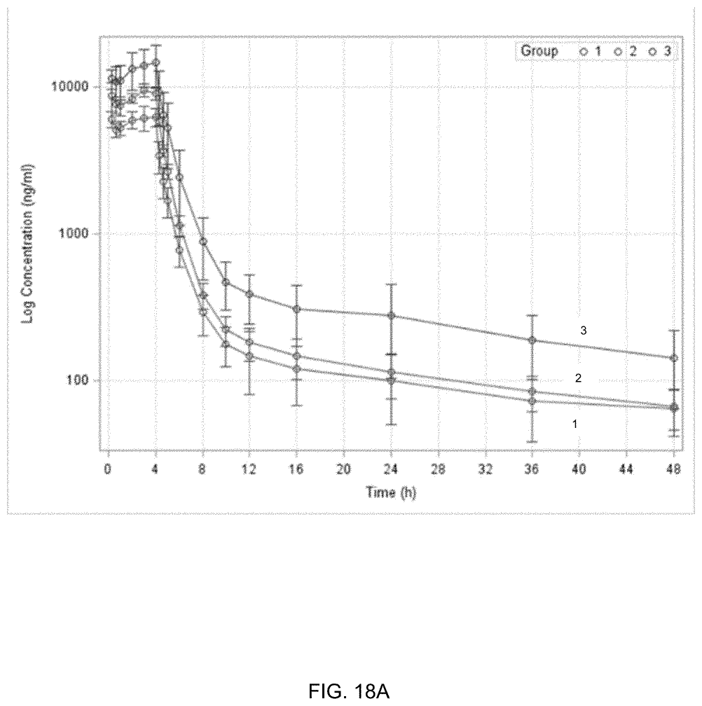

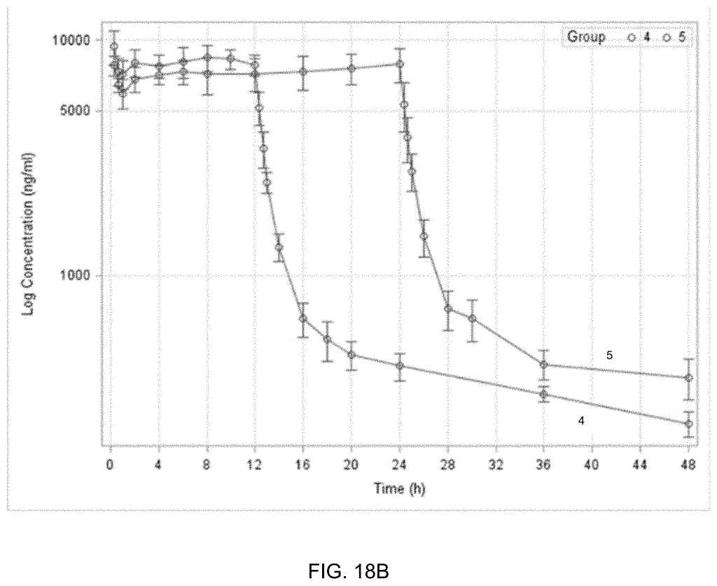

The mean Carbaglu plasma concentrations (±SD) vs. nominal time in group 1, 2, and 3, and 4 and 5 in log-linear scale in the range of time 0 to 48 hours are reported in FIG. 18.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for treating acute hyperammonemia due to hepatic enzyme N-acetylglutamate synthase (NAGS) deficiency, comprising:
identifying a patient as having moderate renal impairment; and
administering carglumic acid to the patient at a daily dosage of 50 mg/kg/day to 125 mg/kg/day divided into two to four doses.

2. A method for treating acute hyperammonemia due to hepatic enzyme N-acetylglutamate synthase (NAGS) deficiency, comprising:
identifying a patient as having severe renal impairment or end stage renal disease; and
administering carglumic acid to the patient at a daily dosage of 15 mg/kg/day to 60 mg/kg/day divided into two to four doses.

3. A method for treating chronic hyperammonemia due to hepatic enzyme N-acetylglutamate synthase (NAGS) deficiency, comprising:
identifying a patient as having moderate renal impairment; and
administering carglumic acid to the patient at a daily dosage of 5 mg/kg/day to 50 mg/kg/day divided into two to four doses.

4. A method for treating chronic hyperammonemia due to hepatic enzyme N-acetylglutamate synthase (NAGS) deficiency, comprising:
identifying a patient as having severe renal impairment or end stage renal disease; and
administering carglumic acid to the patient at a daily dosage of 2 mg/kg/day to 25 mg/kg/day divided into two to four doses.
